(19) United States
(12) Patent Application Publication
(10) Pub. No.: US 2024/0124216 A1
(43) Pub. Date: Apr. 18, 2024

(54) Lockable Electronic Child Resistant Prescribed Medicinal Substance Container

(71) Applicant: Dimitri Leconte Girault, Miramar, FL (US)

(72) Inventor: Dimitri Leconte Girault, Miramar, FL (US)

(21) Appl. No.: 18/220,815

(22) Filed: Sep. 28, 2023

Related U.S. Application Data
(60) Provisional application No. 63/388,194, filed on Jul. 11, 2022.

(51) Int. Cl. B65D 85/10; B65D 43/02; B65D 50/00; B65D 55/02; B65D 77/04; B65D 85/50

(52) U.S. Cl. CPC B65D 85/10; B65D 43/0202; B65D 50/00; B65D 55/02; B65D 77/0486; B65D 85/505; B65D 2543/00092; B65D 2543/00351; B65D 2577/043

(57) ABSTRACT

A lockable Electronic child resistant medicinal substance container is provided and includes a cylindrical medicinal substance container body, a divider ring, a domed medicinal substance container lid, an electromagnetic locking system, a processor, a memory, a battery, a programmable LED light, a wired interface, a wireless interface, and a medicinal substance cartridge assembly having a first and a second sterile medicinal substance cartridges.

### CROSS-REFERENCE TO RELATED APPLICATION

This Non-Provisional Utility application claims the benefit of co-pending U.S. Provisional Patent Application Ser. No. 63/388,194, filed Jul. 11, 2022, which is incorporated herein in its entirety.

### THE FIELD OF THE INVENTION

The present disclosure relates to electronic medicine prescription containers. More particularly, the present disclosure relates to a child resistant smart remotely lockable prescribed cannabis prescription filling, distributing, and storing container and monitoring system which receives and reads barcoded a child resistant cannabis prescription filled cartridges and allows a user to authenticate, convey, secure, and track the child resistant cannabis prescription filled cartridge and the prescribed cannabis or cannabinoid medicinal substance filled in it.

### THE BACKGROUND OF THE INVENTION OF THE INVENTION

The present invention provides a lockable electronic prescribed medicinal substance container. Various child resistant electronic medicinal substance containers are known in the art for use in securing a prescribed medicinal substance by way of electronic lock. These medicinal substance containers are used in pharmacies, assistant living facilities and Dr. Offices etc. These units are large squared, or rectangle tackle box sized distributions stations and they are secured or mounted on a counter or wall. These units are not shaped, sized, or designed to be carried with the patient with an electronic child resistant lid or cap that records the date and time the container is opened by way of sensors on the Lid. These child resistant electronic medicinal substance containers are not designed to be conveyed, tracked, or locked by a peripheral mobile computing device remotely. Also, these child resistant electronic medicinal substance containers are not portable, or pocket sized. They are designed to be for commercial use only. In the medical cannabis industry, there are many counterfeiters who duplicate the packaging of the states licensed medicinal cannabis grower's products. The counterfeiters put cannabis grown on the black market into packaging that looked like the and packaging of the licensed growers. The black market cannabis is not lab tested and often is grown with harmful chemical. The black market cannabis also is often laced with dangerous drugs like fentanyl leading to people overdosing. This creates a deadly hazard for the patients and recreational cannabis consumers. Accordingly, there remains a need for a pocket sized smart lockable medicinal substance container with a medicinal substance monitoring system that can authenticate, convey, track, and secure prescribed sterile medicinal substance cartridges and the medical substances filled in them.

### SUMMARY OF THE INVENTION

The present invention and all the embodiments of the present invention overcomes the deficiencies of the known art and the problems that remain unsolved by providing a smart remotely lockable pocket medicinal cannabis and cannabis product container with a software and computer system which slidable receives and electronically recognizes a pharmaceutical grade, laboratory grade sterile biodegradable child resistant cannabis prescription filled cartridge and, allows a user to authenticate, convey, secure, and track the biodegradable child resistant cannabis prescription filled cartridge and the prescribed medicinal cannabis or medicinal cannabis product filled in it

In accordance with one embodiment of the present invention, the invention consist of a multi-chambered child resistant medicinal substance container comprising, a container body, a the container lid, a medicinal substance cartridge assembly including a first medicinal substance cartridge and a second medicinal substance cartridge, the medicinal substance cartridge assembly designed so that the second medicinal substance cartridge is slidably inserted into the first medicinal substance cartridge, and the whole medicinal substance cartridge assembly can be slidably inserted into the container body, wherein the container lid is secured to the container body via an electromagnetic lock.

In second aspect the child resistant pocket medicinal substance container has a battery and is configured to slidably retain cartridges filled a prescribed medicinal substance.

In third aspect the multichambered child resistant pocket medicinal substance container has a portable medicinal substance container upper end and portable medicinal substance container lower end.

In fourth aspect, the battery is flat and is located at the smart portable electronic medicinal substance container lower end half of the smart container in an electronic elements compartment.

In another aspect the multichambered child resistant pocket medicinal substance container has a medicinal substance container top or a lid and a portable medicinal substance container bottom or a body.

In another aspect, the medicinal substance container body has a cylindrical cavity.

In another aspect, the lid may be separated from the body with a divider ring that rest on and adheres magnetically to a surface of the medicinal substance container body.

In another aspect, the divider ring also rests around a first medicinal substance cartridge when it is inserted into the medicinal substance container body.

In another aspect, any surface on an element or component of the smart medicinal substance container or any element built on the surface of the medicinal substance container may be designed, configured, and orientated to safely provide a power to any other element or device within the medicinal substance container's by making contact to a metallic or magnetic flat surface or male and female configured connectors, plugs, and ports or any other safe way to transfer power from one device to another via a surface, track, or groove known in the art.

In another aspect, the power generated from the metallic or magnetic flat surface or male and female configured connectors, plugs, and ports may power any element of the medicinal substance container that may require power to function.

In another aspect, the multichambered child resistant pocket medicinal substance container may contain every component or feature being hardware or software known in the art to create a fully functional Smartphone for example, Apple's iPhone or Samsung's Galaxy series phones.

In another aspect, the any components or features hardware, or software known in the art at this time or to be known in the future to create a fully functional Smartphone that the smart medicinal substance container may include but is not limited to is a touchscreen, Bluetooth module, Wi-Fi, internet, code scanning features, multiple cameras, mic jacks, mini USB-C connection ports, communication software, multiple microphones, multiple industry leading clear speaker phone quality speaker, touch sensitive touchscreens, sensors, volume button, mute button, passcode screen, fingerprint and face recognition, flashlight, curved shaped glass, GPS, vibrating feature memory, memory card or USB stick style slots or ports, digital scale, digital clocks digital calendars.

In another aspect, the operating system and software of the smart medicinal substance container may be Android or iOS.

In another aspect, the divider ring is configured with multiple programmable LED lights imbedded in, on, or behind a surface of the divider ring and going around the divider ring.

In another aspect, the ring is made of a translucent material and is illuminated by a LED light source shining from behind a surface of the divider ring.

In another aspect the container lid has a top surface and a bottom surface.

In another aspect the medicinal substance container lid and the portable medicinal substance container body each have an upper and a lower end.

A third aspect the container lid has a roof shaped like a dome.

In another aspect, there is a second but different structured container lid that has a top half part and a bottom half part that couple to each other.

In another aspect, the second container lid top half part and bottom half part screw or snap onto each other.

In another aspect, the bottom half part and the top half part are made with a see-through material such as glass or plastic.

In another aspect, any glass surface of any element described herein, such as the containers, packaging, bodies, or cartridges describe herein may be shatter proof, reinforced, plexiglass, and an inch thick or multiple inches thick.

In another aspect, the bottom half part roof is flat and doubles as a cannabis product display pedestal, stage or surface.

In another aspect, the top half part may be a domed or an egg-shaped capsule designed to cover the roof top of the bottom half part of the second lid.

In another aspect, the lid and the body are carved out or hallow and provide a top or lid cavity and a bottom or body cavity.

In another aspect the lid cavity has a lid cavity inner wall and a lid cavity ceiling.

In another aspect the lid has sensors configured with it.

In another aspect the container lid has a speaker configured in it.

In another aspect the container body has a speaker configured to it.

In another aspect the lockable electronic portable medicinal substance container has a first sterile refillable medicinal substance cartridge or chamber and a second sterile refillable medicinal substance cartridge or chamber.

In another aspect, the first sterile refillable medicinal substance cartridge or chamber maybe in a cylindrical, oval, square, rectangle tubular or cigar shape.

In another aspect, any smart medicinal substance cartridge, chamber, packaging bin, bag, or tubes containers described herein may be designed to be inserted in a lockable child resistant smart medicinal substance container.

In another aspect, any child resistant medicinal substance container, bodies, cartridges, chambers, packaging, bins, boxes, bags, crates, tubes, jars, hermetically sealed container, capsule, described herein may be designed to be inserted, placed, slid, pushed, pulled, shuffled, conveyed via a conveyor belt machine and sliding system, rocked into or onto a lockable smart (configured with AI technology) computerized electronic medicinal substance container safe or magnetically locked room.

In another aspect, any child resistant medicinal substance container variation described above or herein disclosed may be configured to moved or be conveyed by the conveyor belt machine and sliding system. The conveyor belt machine and sliding system may be any conveyance machine and system commonly used to move product by any farm, food processing plant, medical substance production plant or industrial factory conveyance system known in the art.

In another aspect, the smart product conveyance system may consist of a frame that supports wheels, rollers, belts configured to move materials.

In another aspect, the conveyor belt machine and sliding system may be a Bucket, Ball Transfer, Vibrating, Vacuum, Pneumatic/Vacuum, Magnetic, Slat/Apron. Powered Roller, Belt, Chute, Wheel, Walking Beam, Screw or Auger, Overhead, Drag/Chain/Tow, or any other conveyer system known in the art.

In another aspect, the conveyor belt machine and sliding system has a frame, the frame may be straight, curved, spiral, Z frames, or any other shaped frame known in the art to for conveyance belt machines and systems frames.

In another aspect, the conveyor belt machine and sliding system may be powered by motor, manually, gravity, magnets, and or electromagnetic system.

In another aspect, the conveyor belt machine and sliding system may be a part of a centralized smart container computer network system designed to move, process, package, label, and electronically classify, profile, lock and track a product produced or derived from any element of a cannabis plant including the seed, bud/flower, leaf, or stem.

In another aspect, any child resistant medicinal substance container variation described above or herein described maybe locked or secured to each other by any method known to secure groups of a product pr containers while being conveyed.

In another aspect, any child resistant medicinal substance container variation described above or herein described maybe designed to be stacked and locked to each other by and means disclosed herein. They may also be locked or stacked on each other by any means known to couple or connect containers, cartridges, capsules, bottles, or vertical standing products in a group together.

In another aspect, the child resistant medicinal substance container variation described above, or any variation herein described maybe manufactured specifically with materials known in the art and in the medical cannabis cultivation industry to be used to grow, store, heat, bake dry, cut, or otherwise process human food, medication, cannabis flower or cannabis extracted products.

In another aspect, any child resistant medicinal substance container variation described above or herein described may be specifically designed to measure and control humidity, measure, and control temperature, vibrate, transport a taxed and government regulated substance, chemical substance, or toxic/hazardous substance, flammable substance, or combustible substance or medicinal substance.

In another aspect, the child resistant medicinal substance container described above, or any variation herein described may be specifically designed and constructed with any food grade materials known known in the art to prevent cross contamination.

In another aspect, the child resistant medicinal substance container smart mentioned above, or any child resistant medicinal substance container disclosed herein may be configured to be sealed with and comply with any or all local, state, or federal government Child-Resistant Packaging (CRP) requirements.

In another aspect the Child-Resistant Packaging requirements may be all (CRP) requirements recognized and enforced by the Poison Prevention Packaging Act (PPPA)

In another aspect, any sterile refillable medicinal substance cartridge packaging described herein or designed to be inserted in the child resistant medicinal substance container may be sealed by a pry-off metal crown cork-style bottle or container cap/lid (for packaging or cartridges containing a single serving.

In another aspect, any sterile refillable medicinal substance cartridge packaging described herein or designed to be inserted in the child resistant medicinal substance container may be plastic packaging that is a least 4 mils thick and heat-sealed without an easy-resistant open tab, dimple, corner, flap (packaging of cannabis goods that qualify for single-use CRP or that contain only a single serving).

In another aspect, the term “Single Use” (“Initial CRP”) may refer to packaging or a cartridge that is initially child resistant, but once it is opened, it is no longer child resistant. If used, the packaging or cartridge label may have a warning informing the customer or patient that the “package is not child resistant once the seal is broken and the package or the cartridge is opened.

In another aspect, the term “Multiple Use” (“Lifetime CRP”) may refer to a container, packaging, or cartridge that is Child-Resistant throughout the life of the package. This packaging may be opened and resealed or closed and still retain its Child-Resistant qualities and or features.

In another aspect, the term “Single Use” may refer to the use of Cannabis Flower, Pre-rolled Cannabis Cigarettes, Topicals, Dabs, Shatter, Wax, Vaping Oil Solutions, and the like.

In another aspect, the term “Multiple Use” may refer to Cannabis infused edibles, orally consumed concentrates such as capsules, tinctures, and the like.

In another aspect, the child resistant medicinal substance container and any refillable medicinal cartridge disclosed herein or designed to be inserted in the child resistant medicinal substance container may contain and legally seal Cannabis Flower, Pre-rolled Cannabis Cigarettes, Topicals, Dabs, Shatter, Wax, Vaping Oil Solutions in a method meeting and complying with the Poison Prevention Packaging Acts (PPPA).

In another aspect, the child resistant medicinal substance container and any refillable medicinal cartridges disclosed herein or designed to be inserted in the child resistant medicinal substance container may be labeled lawfully and in compliance with all state, local, and federal laws/requirements for labeling a prescribed narcotic or product produced by any element of a cannabis plant.

In another aspect, the government complying label is an Identification sticker marked with a barcode.

In another aspect, the government complying label may be configured with a RFID chip.

In another aspect, the information on the government complying label designed to be applied to any smart container disclosed herein may be accessed by scanning the RFID Chip or Barcode with a reader/scanner.

In another aspect, the packaging or cartridge has a “Primary Label”, or the part of the label easily displayed to the customer when in the product display case. The “Primary Label” may be on the top of or in front of the refillable medical container, refillable medical cartridge, bin, box, or packaging.

In another aspect, the child resistant medicinal substance container has its own “Primary Label”, or the part of the label easily displayed to the customer or distributer when in a product display case. The “Primary Label” may be on the top of the child resistant medicinal substance container.

In another aspect, any label describe herein may be applied on and adhered to the first sterile refillable medicinal substance cartridge and a second sterile refillable medicinal substance cartridge or chamber. The surface of any container, cartridge, packaging, display case, docking station or any other physical element on the hardware disclosed herein.

In another aspect, all barcoded labels to be placed on the child resistant medicinal substance container or on the first sterile refillable medicinal substance cartridge or the second sterile refillable medicinal substance cartridge may list multiple facts about the medicinal substance that may be contained in the first sterile refillable medicinal substance cartridge or the second sterile refillable medicinal substance cartridge. Barcoded Labels may identify the medicinal substance product by known name or common name, list weight of the medicinal substance the first sterile refillable medicinal substance cartridge or the second sterile refillable medicinal substance cartridge contains in both metric and us units, be branded with a government required symbol or logo, display an UID number or other required Unique Tracking ID or number, display the government appointed license number of the cannabis medicinal substance cultivator, grower, packager, distributer or dispensary, display the date the medicinal substance was packaged in the first sterile refillable medicinal substance cartridge or the second sterile refillable medicinal substance cartridge including day, month and year, display government warning statement, display all or some of the lab results including the percentages of the THC, CBD, CBG, or other known or unknown cannabinoid molecules naturally found in the cannabis plant.

In another aspect, any information and facts required by law to be on the labels of medically prescribed cannabis substance containers or packaging may be uploaded to the centralized smart container computer network data base and stored on in a memory. This information may be accessed by scanning the RFID tag or Barcoded Label placed on the first sterile refillable medicinal substance cartridge or the second sterile refillable medicinal substance cartridge. The internet connected smart container centralized computer network is designed to identify, track, manage, weigh, test, locate, lock, or unlock the first sterile refillable medicinal substance cartridge or the second sterile refillable medicinal substance cartridge that are inserted and secured in the child resistant medicinal substance container.

In another aspect, the child resistant medicinal substance container and the first sterile refillable medicinal substance cartridge or the second sterile refillable medicinal substance cartridge may be any color, including but not limited opaque, clear, red, yellow, blue, purple, green, orange, gold, brown, tan, sky blue, baby blue powder blue, turquoise, amber, pearl, lavender, or pink.

In another aspect, the child resistant medicinal substance container and the first sterile refillable medicinal substance cartridge or the second sterile refillable medicinal substance cartridge may be any size or shape known to exist including but not limited to cylindrical, square, rectangle, oval, spiral, octagon, pentagon, triangle, circular, diamond, Donut or any other shape.

In another aspect, the child resistant medicinal substance container, or any sterile refillable medicinal substance cartridge described herein may be marked with a licensed NFL, NHL, NBA, or MLB team logo brand or mascot.

In another aspect, there may be the child resistant medicinal substance container may configure to be shaped like a large Rubbermaid style plastic or rubber bin. The Rubbermaid bins configured with the same electronic hardware and/or software that cylindrical shaped child resistant medicinal substance container may be configured with.

In another aspect the first sterile refillable medicinal substance cartridge assembly is designed to be slidably inserted into the upper end of the cylindrical medicinal substance container body and retained by the child resistant medicinal substance container body cavity.

In another aspect once fully inserted into the container body cavity of the cylindrical medicinal substance container body, the first sterile refillable medicinal substance cartridge is designed to make a clicking or snapping sound when connecting and locking in fully in place like a seat belt being secured in a car.

In another aspect once the first sterile refillable medicinal substance cartridge is fully inserted into cylindrical medicinal substance container body cavity, the second sterile refillable medicinal substance cartridge is designed to be slid and inserted into cavity of the first sterile refillable medicinal substance cartridge and make a clicking or snapping sound and connect and lock in place like a seat belt being secured in a car.

In another aspect, the child resistant medicinal substance container may also make a beeping sound via a micro speaker or flash a LED Lights in a certain pattern when a sterile refillable medicinal substance container cartridge is snapped in fully inserted the medicinal substance container body.

In another aspect the first sterile refillable medicinal substance cartridge has an insertable main container chamber upper end and an insertable main container chamber lower end.

In another aspect the sterile refillable medicinal substance cartridge assembly may have multiple medicinal substance cartridges with different shapes and sizes.

In another aspect the sterile refillable medicinal substance cartridge assembly may be made from stainless steel or carbon fiber.

In another aspect the sterile refillable medicinal substance cartridges may have different shaped cavities for example spiral chamber, square shaped chamber, cone shaped chamber with the peak of the cone facing up or facing down.

In another aspect the electronic portable medicinal substance container provides a second sterile refillable medicinal substance cartridge.

In another aspect the second sterile refillable medicinal substance cartridge contains multiple sterile medicinal substance chambers.

In another aspect the multiple sterile medicinal substance chambers are shaped in a cylindrical or tubular shape.

In another aspect the second sterile refillable medicinal substance cartridge chambers are designed to receive and retain a medicinal or physician prescribed substance.

In another aspect the medicinal or physician prescribed substance is a substance that contains at least one element produced from a plant or tree.

In another aspect the plant may be a mushroom, soursop tree, cannabis plant, or opium poppy plant.

In another aspect the medicinal substance is a cannabis or mushroom extract.

In another aspect the substance derived from the cannabis plant is the bud or flower produced by the cannabis plant.

In another aspect the cannabis plant is patented or grown with a patented method.

In another aspect the medicinal substance is a plant extract product such as a wax, butter, oil, lotion, candy, topical or other product known to be made with or infused with a molecule derived from a cannabis or opium poppy plant.

In another aspect the second sterile refillable medicinal substance cartridge is shaped and designed to receive and retain multiple pre rolled cannabis cigarettes.

In another aspect the second sterile refillable medicinal substance cartridge may have a head shaped and configured like a cylinder of a six-shooter revolver pistol configured with six head holes/ports leading to six medicinal substance chambers/tubes The holes designed to receive and retain pre-rolled cannabis cigarette.

In another aspect the pre rolled cannabis cigarettes are rolled by hand or a joint rolling machine apparatus.

In another aspect, the first and second medicinal substance or pre rolled cannabis cigarettes are rolled with color coordinated cigarette paper to identify a type or classification of a medicinal substance rolled in it.

In another aspect the pre rolled cannabis cigarettes are rolled with a tobacco leaf, hemp paper, rice paper, blunt or cigar paper or any other leaf or material used to roll tobacco or cannabis substances.

In another aspect the tobacco leaf, hemp paper, rice paper, blunt paper used to pre rolled cannabis cigarettes above may be colored red, blue, yellow, green, blue, brown, white, black, purple, lavender, orange, sky blue, burgundy, or any other color known to exist.

In another aspect, the colors disclosed above may signify THC CBD or CBG percentage potency, identify the cannabis cultivator, identify State jurisdiction, identify dispensary brand, signify if the cannabis product is derived from sativa or indica strain.

In another aspect the pre rolled cannabis cigarettes are rolled with papers that have segmented lines and or marks like the lines on a ruler that designate indicate the percentage or dosage of a particular substance rolled or contained within said designated lines sections once the cannabis cigarette is rolled.

In another aspect the pre rolled cannabis cigarettes are rolled with papers that are marked with lines segmenting the cannabis cigarettes in quarters, thirds, or halves.

In another aspect any of the medicinal substance disclosed or mentioned herein may have a commonly known or branded name.

In another aspect, the multiple sterile medicinal substance chambers are cylindrical and elongated and have unscrew able or detachable base segments for cleaning ash out from lit cannabis cigarettes put into the chambers when still lit to be extinguished.

In another aspect, the segmented bases of the chambers may be configured with an ash tray/trap or other crumbling or falling material traps.

In another aspect, a grilled or perforated divider covers, or caps cover the ash trap and separates the main cavities of the chambers from their base ash traps. The grilled or perforated dividers aiding in brush against the cannabis cigarette's lit side scrubbing and smoothing the tip of the cannabis cigarette for lighting the cigarettes again at another time.

In another aspect, the first and second medicinal substance container cartridges are color coordinated to identify a type or classification of a medicinal substance.

In another aspect the child proof locking mechanism assembly is made up of elements located on the first or second sterile refillable medicinal substance cartridges, the container body, and the container lid.

In another aspect the child proof locking mechanism assembly is made up of a two-dimensional nib sliding channel locking system.

In another aspect the two-dimensional nib slide channel locking system is part of a magnetic locking mechanism assembly that includes at least one magnet.

In another aspect the two-dimensional nib sliding channel locking system is made up of a two-dimensional nib sliding channel, a first and second nib channel sliding entrance, a first and second nib key, a vertical nib channel portion, a circular nib channel portion and a series of strategically placed magnets along the two-dimensional nib sliding channels and electromagnetic locking system.

In another aspect the t electromagnetic locking system include an electromagnetic or electromagnets positioned directly under, or behind the surface of the two-dimensional nib sliding channel.

In another aspect the two-dimensional nib slide channel system may have metallic surfaces configured to contact with multiple magnetized or magnetic nib keys.

In another aspect the nib channel keys may be located somewhere on the upper end of the outer wall of the first refillable medicinal substance cartridge.

In another aspect the nib channel keys may be located somewhere on the lower end of the outer wall of the first refillable medicinal substance cartridge.

In another aspect the nib keys are sized and shaped to be inserted into the nib channel slide entrances.

In another aspect the nib keys are sized and shaped to securely slide on the two-dimensional nib sliding channels.

In another aspect, the nib keys may be located on an element of the medicinal substance container body instead of the first refillable substance cartridge. In this way the medicinal substance container may be sealed with or without the first refillable medicinal substance container being inserted in the container body cavity.

In another aspect, the electromagnetic locking system magnets are configured to interact with the magnetic nib keys, so they attract or repel each other stopping or resisting the nib keys from being slid up, down or around the two-dimensional nib sliding channel without a force stronger than the pulling or repelling force generated from the magnets.

In another aspect, the electromagnetic locking system system's magnets are configured to use their magnetic forces (attraction and or repulsion) to aid or enable pulling or pushing of an element or elements of the locking mechanism assembly or the two-dimensional nib sliding channel system to aid in the turning or resist the turning of the container lid.

In another aspect the electromagnetic locking system electromagnets and or magnetized surfaces are connected to a wired circuit or circuits that may send or flow electrical current to the magnets or magnetized surfaces disclosed herein to amplify or decrease their magnetic attraction or repulsion force thereby locking or unlocking the medicinal container lid to the medicinal substance container body.

In another aspect the two-dimensional nib sliding channels are sized and shaped or otherwise configured to securely receive, retain, guide, and magnetically attract or repel the container nib keys.

In another aspect the container lid may provide the two nib channel slide entrances at the bottom end of the container lid.

In another aspect the container lid may provide two nib channel slide entrances at the top end of the container lid.

In another aspect the vertical nib channel portions are carved out of the bottom surface of the container lid and the inner wall of the container lid. The vertical nib channel portions extending vertically from the bottom end of the container lid toward the top end of the lid.

In another aspect the vertical nib channel portion extends vertically until it meets with the circular nib channel portion carved into the inner wall of the container lid forming the two-dimensional nib channel slide. The circular nib channel portions are also sized and shaped to receive and engage with the two nib keys.

In another aspect the strategically placed electromagnets are placed on the nib keys and on the two-dimensional nib channel slide channels (circular or vertical channel portions).

In another aspect the strategically placed electromagnets are placed within the nib keys and within the two-dimensional nib channel slide.

In another aspect, the strategically placed electromagnets are placed within the two nib channel slide entrances to attract and guide the nib keys toward the two nib channel slide entrances.

In another aspect the nib keys are shaped and sized to be inserted into the nib channel slide entrances and slide vertically into and onto the two-dimensional nib channel slide.

In another aspect the electromagnetic lock and security system is an electronically managed system.

In another aspect the electromagnetic locking mechanism assembly consist of high-powered electromagnets, connection wires, metal magnet engagement plates, a switch or button and an electronic system to reverse the electromagnets polarity.

In another aspect the electromagnetic locking mechanism assembly contains multiple electromagnets.

In another aspect the electromagnetic locking mechanism assembly contains two electromagnets shaped like a ring with 90-degree edges and flat surfaces. The two ring shape electromagnets configured to two separate elements of the medicinal substance container.

In another aspect, one of the two ring shape electromagnets configured to be placed into or under a surface of the lid of child resistant medicinal substance container. The other ring-shaped electromagnets is configured to be placed into or under a surface of the cylindrical shaped body of the medicinal substance container or the container divider ring between the container lid and the cylindrical shaped body of the child resistant medicinal substance container.

In another aspect, the magnetic field or pull or push force of the electromagnets can be amplified, decreased, or reversed with the touchscreen interface of a peripheral smart mobile computing device such as a smart phone, tablet, or laptop computer paired with the child resistant medicinal substance container that has downloaded and installed the smart child resistant medicinal substance container custom software application designed for the smart electronic handheld portable medicinal substance container and its systems.

In another aspect, the electromagnetic locking mechanism assembly may be configured to contain an electromagnet and armature plate.

In another aspect the electromagnetic locking mechanism assembly may be configured in or with the two-dimensional nib sliding channel system.

In another aspect the electromagnetic locking mechanism assembly may configured to contain an electromagnet and armature plate designed and orientated to be attracted to each other strongly enough to keep the container lid from being rotated or removed.

In another aspect, any magnet or magnetized surface of the electromagnetic locking mechanism assembly or any electromagnet or magnetized surface disclosed herein may be replaced with a metallic armature plate.

In another aspect the electromagnetic locking mechanism assembly may configured to be a “Fail Safe” electromagnetic locking system where power is required to lock the electronic medicinal substance container lid to the lockable handheld electronic medicinal substance container.

In another aspect the mag electromagnetic locking mechanism assembly may configured to be a “Fail Secure” electromagnetic locking system where power is required to unlock the electronic medicinal substance container lid from the lockable handheld electronic medicinal substance container.

In another aspect, the electromagnetic locking mechanism assembly may be configured with a small or micro metal “latch”, “bar”, “Bolt”, or “nib” (small metal protruding element) and a “strike plate” (a metal plate or assembly with a “latch” or “bolt” receiving hole). The latch or strike plate may be installed on or within any surface of any element of the electronic medicinal substance container disclosed herein.

In another aspect, the ma electromagnetic locking mechanism assembly may be configured with a small or micro “strike plate” installed on or within the surface of the two-dimensional nib sliding channels. The strike plate may be aligned, configured, and designed to receive the “Latch”.

In another aspect, the electromagnetic locking mechanism assembly may be configured with a small or micro metal retractable “latch” “bar” or nib (small metal protruding element) may be located on the first sterile refillable medicinal substance cartridge upper end (where the two nib sliding keys are located).

In another aspect, the two latches that may be located on the first sterile refillable medicinal substance cartridge upper end (where the two nib sliding keys are located) are designed to be magnetically pushed or pulled into the “strike plate” and or designed to magnetically pushed or pulled out of the “strike plate”.

In another aspect, power may be supplied to the “strike plate” to keep the “latch” or lock bolt in place once inserted in the “Strike Plate” keeping the container lid locked onto the container body and first sterile refillable medicinal substance cartridge.

In another aspect, any metallic armature plate, or metallic or armature plated surface of the electromagnetic locking mechanism assembly, or two-dimensional nib sliding channel system may be replaced or exchanged with a magnet or magnetized surface or vice versa.

In another aspect, the latch release system or any other element or function of the electromagnetic locking mechanism system may be controlled electronically by way of any user interface or touchscreen user interface disclosed herein. The user interface or touchscreen user interface sending signals to the processor to lock or arm the electromagnetic lock.

In another aspect, user interface or touchscreen user interface may be located on the electronic handheld portable medicinal substance container.

In another aspect, the user interface or touchscreen may be located on a peripheral smart mobile computing device such as a smartphone, smartwatch, tablet, or laptop.

In another aspect, the user interface or touchscreen user interface may be a remote control with a touchscreen user interface designed and paired with to specifically control or manipulate any feature of the child resistant smart electronic pocket portable medicinal substance container.

In another aspect, any configuration, orientation, position, or material of the elements used in the electromagnetic locking mechanism and two-dimensional nib sliding channel system disclosed herein may be switched, reversed, exchanged, or interchanged or substituted with each other to improve on the designs function and improve the user experience.

In another aspect the electronic child resistant medicinal substance container may have a tray style docking station where the electronic child resistant handheld portable medicinal substance container can be docked and charged.

In another aspect the docking station has a backup battery.

In another aspect the docking station has multiple connectors and/or a connection ports.

In another aspect, a showcase display style docking station for commercial use can display, charge and program multiple smart child resistant electronic medicinal substance containers at the same time.

In another aspect the display style docking stations can be designed to be placed in a traditionally used see through or glass counter product display case.

In another aspect the electronic handheld portable medicinal substance container has multiple connection ports and/or a connector.

In another aspect the connector and/or the connection port of the docking station and the connection port and/or the connector of the electronic portable medicinal substance container are designed to engage with each other.

In another aspect the docking station has an electronic handheld portable medicinal substance docking receptacle or receptacles.

In another aspect the connector and/or the connection port is located within the docking receptacles.

In another the aspect the connector and/or the connection port is a USB style charging port.

In another the aspect the connector and/or the connection port is a USB style data port

In another the aspect the docking station and the electronic medicinal substance container have each a wireless charging feature.

In another aspect the wireless charging feature is embedded under, in or on the docking receptable or receptacles surface.

In another aspect the wireless charging feature is embedded under, in, or behind a surface of the electronic medicinal substance container.

In another aspect the wireless charging feature is embedded under, in, or behind the base surface of the electronic medicinal substance container.

In another aspect medicinal substance container wireless charging feature is embedded under, in, or behind the medicinal substance container body wall surface of the electronic medicinal substance container.

In another aspect, the lockable electronic handheld medicinal substance container may be charged laying horizontally or standing vertically on the charging display docking station.

In another aspect the docking station receptacles may shaped circular and designed to receive the circular container body base of the handheld medicinal substance containers while charging the lockable electronic handheld medicinal substance containers.

In another aspect the docking station receptacles are elongated and shaped like a half cylinder and designed to receive the lockable electronic handheld medicinal substance containers horizontally while charging the lockable electronic handheld medicinal substance containers.

In another aspect the docking station or stations are configured to send and receive Wi-Fi, Bluetooth, RFID or any other signal or data transferring device or signal communicating system known in the art.

In another the aspect the docking station and the medicinal substance container have each multiple LEDs used to indicate at least one of the following, battery power status, locked or unlocked status of the medicinal substance container, Wi-Fi, and Bluetooth connectivity status, the type of substance contained in the container, a specific brand or quality associated with a particular brand, strain or type of cannabis.

In another aspect, the container may have LED lights may be located on the lid, body, divider ring or cartridges of the lockable electronic handheld portable medicinal substance containers.

In another aspect the LED lights may be configured or programed to display each the same color or each different color simultaneously.

In another aspect one LED's may be configured to flash or create light shows.

In another aspect the lights may indicate that the cannabis product in the container is from sativa, indica, or a hybrid strain of cannabis.

Introducing another embodiment, a lockable electronic handheld portable medicinal substance container with a smart container management computer system with a touchscreen, a light sensors, movement sensors, sound sensors, a smart refillable medicinal substance cartridge assembly, a child proof locking mechanism assembly, a biometric authentication feature and an electronic security system managed by a microcontroller operating a custom system software application designed to integrate the lockable electronic handheld portable medicinal substance container's system with a system of a peripheral device or multiple devices.

In another the aspect, the child resistant portable medicinal substance is configured to store data and send and receive encrypted data signals in a HIPPA approved and compliant method.

In another the aspect the child resistant portable medicinal substance is configured to be remotely locked and unlock using a feature of the system of the peripheral device or devices via a communication link (Wired or Wireless).

In another the aspect, the peripheral device or devices may be a remote controller, a computer, or a portable computing device such as a smartphone, a tablet, or a laptop computer paired or synced via a communications link.

In another aspect, the computer processor (CPU) is configured to receive signals sent from the user interface on the peripheral smart computing device or receive signals from the touchscreen user interface on the child resistant medicinal substance container.

In another aspect, the child resistant portable medicinal substance's computer processor may be an element of a microcontroller.

In another aspect the child resistant portable medicinal substance may have a user interface.

In another aspect the user interface may be a touch panel interface.

In another aspect a touch panel interface may be a touchscreen interface.

In another aspect, the interface may provide a passcode screen to unlock the container and enter its data base.

In another aspect the child resistant portable medicinal substance container may have a second user interface, the second user interface being a fingerprint scanner or reader.

In another aspect the child resistant portable medicinal substance container's system may have a retina or eye scanner/reading device.

In another aspect the child resistant portable medicinal substance system may have a face recognition technology.

In another aspect the child resistant portable medicinal substance container may have a third user interface, the third user interface being a user interface provided by the peripheral device.

In another aspect the user interface provided by the peripheral device may be used to remotely lock, unlock, or locate the child resistant portable medicinal substance container.

In another aspect the child resistant portable medicinal substance container is configured to store data on a memory device.

In another the aspect the child resistant portable medicinal substance container is configured send and receive data signal to be processed and/or stored.

In another the aspect the medicinal substance monitoring system is configured to process entries from a digital keypad.

In another the medicinal substance monitoring system enables the user to record a date and a time the portable medicinal substance container has been opened, locked, or unlocked.

In another the aspect the medicinal substance monitoring system is configured to display a real time status of the portable medicinal substance container.

In another the aspect the real time status is at least one of the following a percentage of battery power, location, opened or closed status of the medicinal substance container, or a locked or a unlocked status of the medicinal substance container.

In another aspect the pressure sensor is configured to detect the weight of the first and second sterile medicinal substance cartridges.

In another aspect a light sensor is configured to detect light or lack of light in the environment of the medicinal substance container and send a data signal to the processor.

In another aspect a vibration sensor is configured to detect any movement of the medicinal substance container and send a data signal to the processor.

In another aspect the data received from sensors is used to regulate the amount of power and the time that the battery of the child resistant medicinal substance container utilizes for its system to power the electromagnetic locking system.

In another aspect the medicinal substance monitoring system is configured to put the child resistant medicinal substance container into sleep mode or battery saving mode depending on data signals received by its sensors and sent the processor to be read. The medicinal substance monitoring system having a pocket or purse mode wherein the light sensor, the vibration/motions sensor, and the sound sensors temporarily turned off when the child resistant medicinal substance container is being conveyed in a user's pocket or purse, so the battery has a longer power life.

In another aspect the child resistant medicinal substance container is configured to keep record of the weight and identity of the medicinal substance it retains.

In another aspect the child resistant medicinal substance container is part of and can connect to the smart container centralized network and data cloud system designed to electronically tag, track, monitor, keep data on containers with the GPS module and the sensors.

In another aspect element of the child resistant medicinal substance container is constructed with wood or precious metals.

In another aspect the wood may be bamboo, mahogany, oak, pine, maple, or any wood known to man.

In another aspect the precious metal may be gold, silver, platinum, coper, or any other metal known to man.

### BRIEF DESCRIPTION OF THE DRAWINGS

FIG. 1 presents a front view with the smart lockable electronic medicinal substance container locked and assembled.

FIG. 2A presents a top front left view of the medicinal container divider ring.

FIG. 2 B presents a bottom front left view of the medicinal container divider ring, and a top front left view of the medicinal container body. FIG. 2 B presents the medicinal container divider ring being placed on the upper end of the medicinal container body. The FIG. clearly displays the rail channel and the ring support rail of the medicinal container divider ring.

FIG. 3 A presents an exploded top front left view of the order of assembly/STEPS to assemble the container body, the divider ring and the first medicinal cartridge.

FIG. 3 B presents a bottom front left view of the medicinal substance container body.

FIG. 4 A presents a top front left view of a step to assemble the refillable medicinal substance cartridge assembly. The FIG. A shows the downward motion reference arrows displaying the step to insert the second refillable medicinal substance container into the first refillable medicinal substance container.

FIG. 4 B presents a top front left view of the second refillable medicinal substance container fully inserted into the first refillable medicinal substance container. The FIG. further presents the upward motion reference arrow show the direction of its removal.

FIG. 5 presents a bottom front left exploded view the steps to assemble the lockable electronic medicinal substance container. The FIG. 5 shows the downward motion reference arrows displaying the motion and the steps to place the elements onto and into each other and fully assemble the lockable electronic medicinal substance container.

FIG. 6 A presents a top front left view of the medicinal substance container with the container lid or cap removed.

FIG. 6 B presents a Bottom front left view of the medicinal substance container with the container lid or cap removed.

FIG. 7 presents a top view of the electronic medicinal substance container with the Lid or cap removed showing the top view of the refillable medicinal substance cartridge assembly and a top of a pair of nib keys.

FIG. 8A presents a top front left view of the substance container unlocked with the orientation status marker aligned and a motion direction reference arrow showing a direction that the container lid may be turned to lock the container.

FIG. 8B presents a top front left view of the substance container locked unlocked with the orientation status marker unaligned and a motion direction reference arrow showing two directions that the container lid may be turned.

FIG. 9A presents a bottom left front view of the container lid. The FIG. 9A shows partial of the two-dimensional nib sliding channel, surface C and B of the second right vertical nib sliding portion.

FIG. 9B presents a bottom right front view of the container lid. The FIG. 9B shows partial of the two-dimensional nib sliding channel, surface A and C of the first left vertical nib sliding portion.

FIG. 10A presents a back bottom right view of the container lid. The FIG. 10A shows partial of the two-dimensional nib sliding channel, surface C and B of the first left vertical nib sliding portion, and surface A of the second right vertical nib sliding portion, and the surface A and C of the circular nib channel sliding portion.

FIG. 10B presents a back bottom left view of the container lid. The FIG. 10B shows partial of the two-dimensional nib sliding channel, surface A and C of the second right vertical nib sliding portion, and surface A of the first left vertical nib sliding portion, and the surface A of the circular nib channel sliding portion.

FIG. 11 FIG. 1 presents a front view of another embodiment of a smart lockable electronic medicinal substance container with a touchscreen locked and assembled.

FIG. 12 presents a Bottom front left view of the smart medicinal substance container with the container lid or cap removed and a downward pointing motion reference arrow.

FIG. 13A presents a bottom front left view of a medicinal container divider ring with Colored LED Lights embedded on its surface, and a top front left view of the medicinal container body.

FIG. 13B presents the medicinal container divider ring with programmable Colored LED Lights embedded on its surface being placed on the upper end of the medicinal container body. The FIG. clearly displays the rail channel and the ring support rail of the medicinal container divider ring.

FIG. 14 presents a bottom front left exploded view the steps to assemble the smart lockable electronic medicinal substance container with a touchscreen. The FIG. 14 shows the downward motion reference arrows displaying the motion and the steps to place the elements onto and into each other and fully assemble the lockable electronic medicinal substance container.

FIG. 15 presents a partial front view of the cylindrical container body lower half showing the Status LED Lights, the Speakers, and the speaker grill.

FIG. 16 presents a flow chart for a smart container electronic magnetic locking system.

FIG. 17 presents a flow chart of the functions of the smart lockable portable medicinal substance container of FIG. 1 land the peripheral smart mobile computing device.

FIG. 18 presents a flow chart of the process of how information of a cannabis harvest is entered into the smart container centralized network. The Cannabis Plants are harvested, weighed, tested, and lab results are acquired. The Harvest date, cannabis strain, lab results and other all data about the harvest are recorded. The Barcoded labels are created and programed with the harvest data is recorded onto the bar code. The Barcoded labels are then placed on and adhered to the first and second sterile medicinal substance cartridges. The first and second sterile medicinal substance cartridges are filled with a prescribed cannabis substance and flower to be distributed and monitored by the smart container centralized computer network and data cloud system in sync with the medicinal substance monitoring systems.

FIG. 19A presents a front top view of a first cylindrical substance chamber tube with a smart container centralized network law compliant barcoded label used to package a cannabis cigarette and can screw into the second medicinal substance cartridge head.

FIG. 19B presents a front top view of the second medicinal substance cartridge head with two smart container centralized network law compliant barcoded labels adhered to the first and a second cylindrical substance chamber tube used to package cannabis cigarettes screwed into to the second medicinal substance cartridge head.

FIG. 19C presents a front top view of the first sterile medicinal substance cartridge with the smart container centralized network law compliant barcoded label adhered to it. The first sterile medicinal substance cartridge used to package flower or other cannabis product and may be sealed with single or multiple use child resistant methods.

FIG. 20 presents the cannabis medicinal substance RFID tracking, monitoring, identify system that may be used to monitor cannabis smart container's their contents, and the employees hired to manage them and the facilities they are managed at when synced to the smart container centralized computer network and data cloud system and the medicinal substance monitoring system.

Since many modifications, variations, and changes in detail can be made to the described preferred embodiments of the invention, it is intended that all matters in the foregoing description and shown in the accompanying drawings be interpreted as illustrative and not in a limiting sense. All parts, features, systems, networks disclosed herein may be interchanged, used with all and any embodiments disclosed herein. Thus, the scope of the invention should be determined by the appended claims and their legal equivalence.

### DETAILED DESCRIPTION OF REPRESENTATIVE EMBODIMENTS

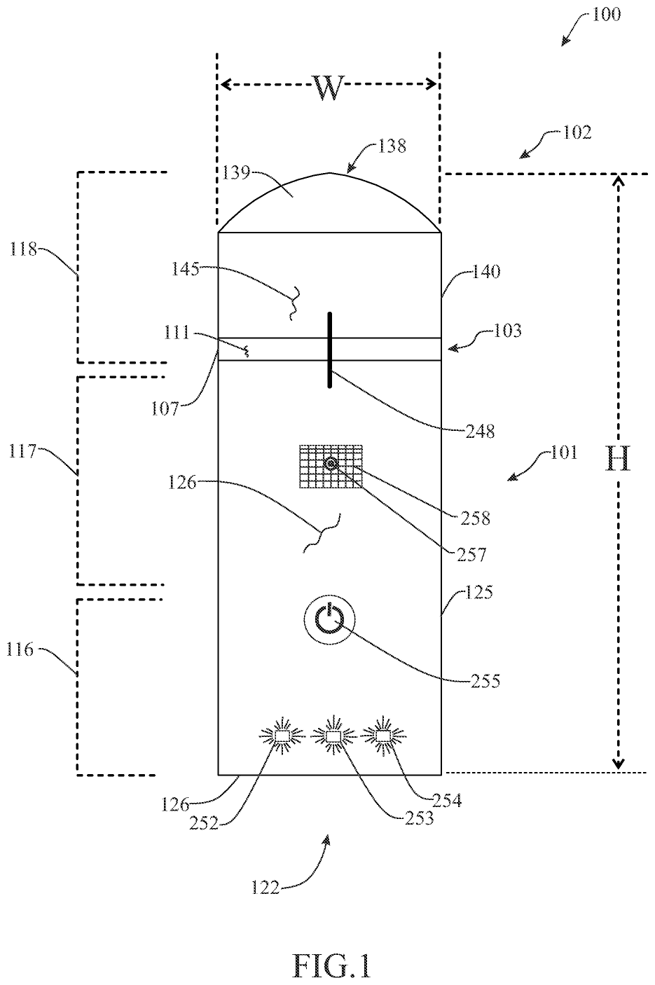

The following detailed is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “back” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

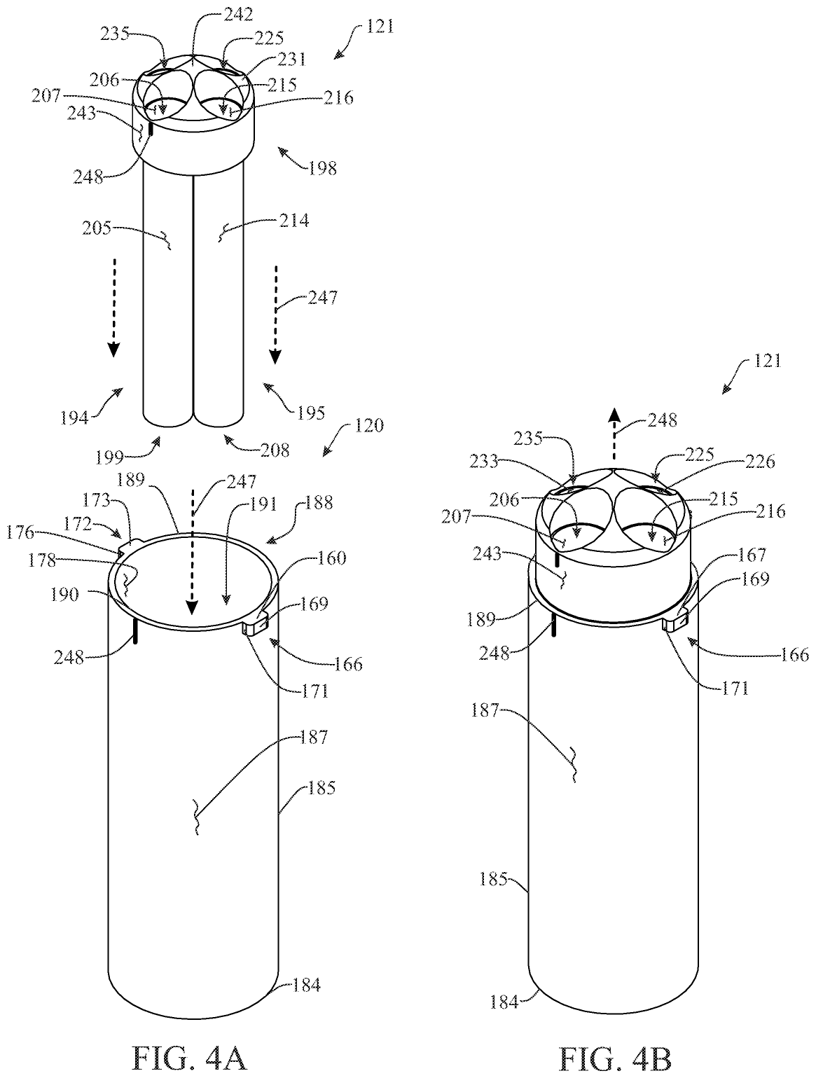

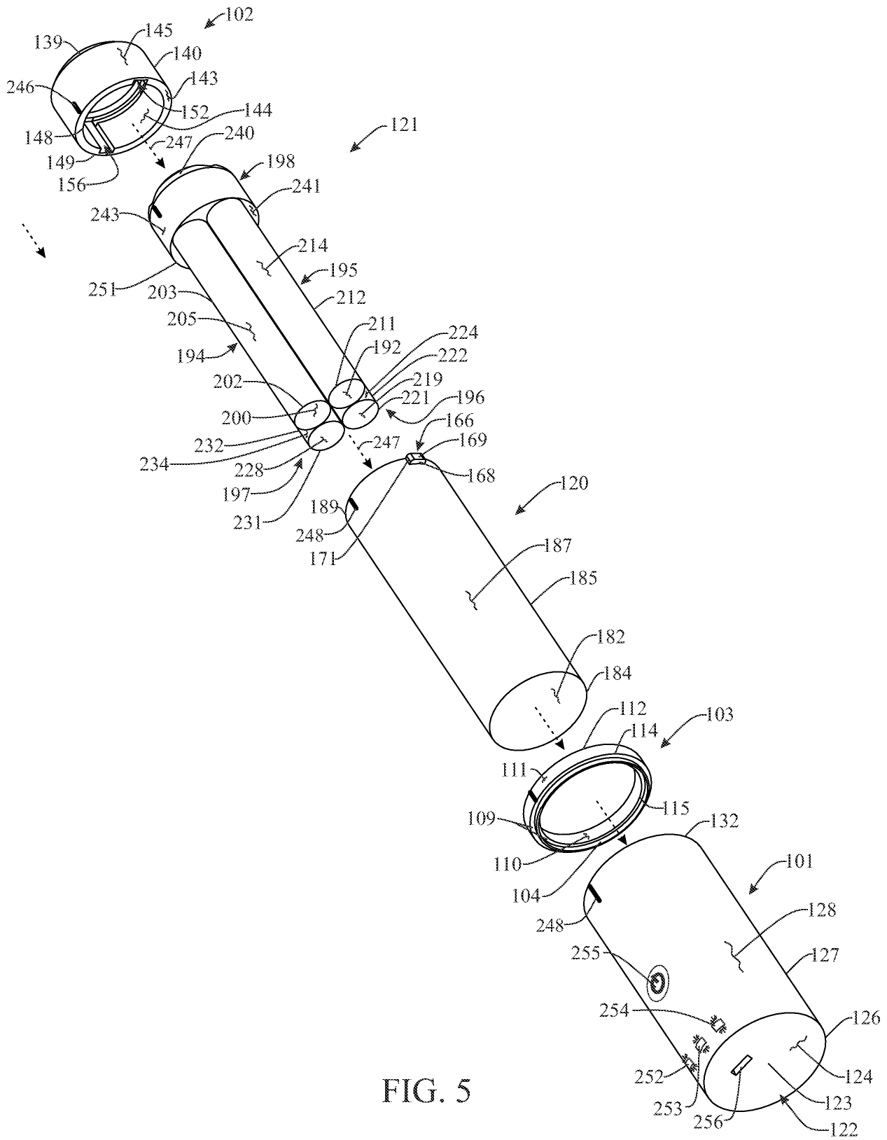

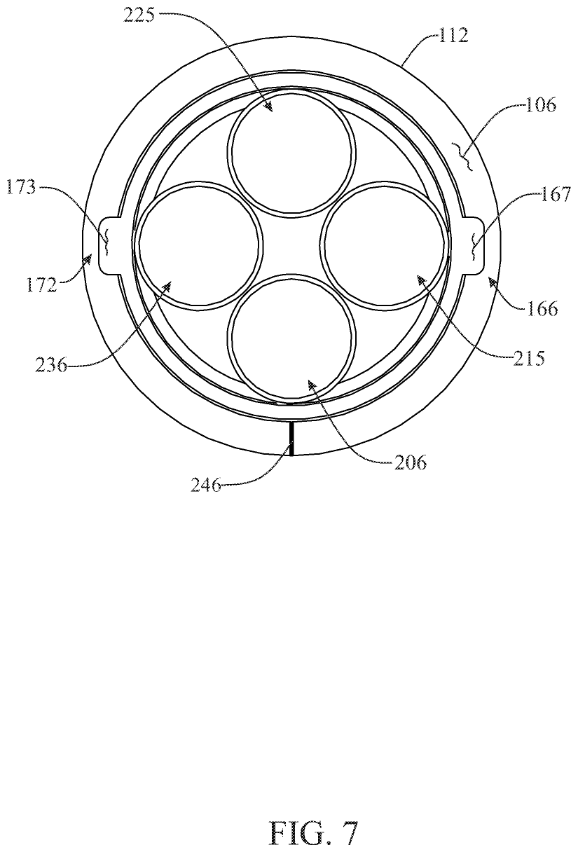

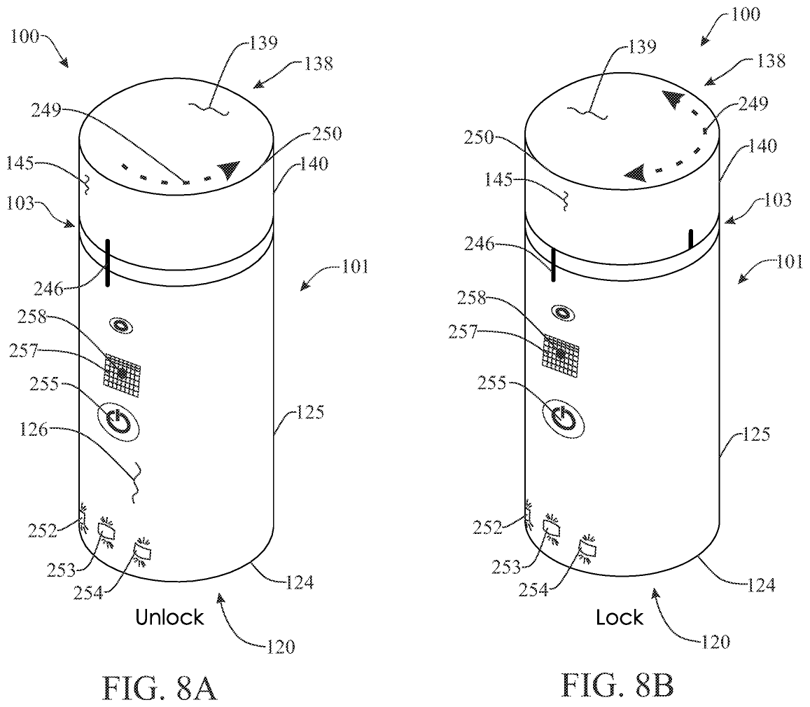

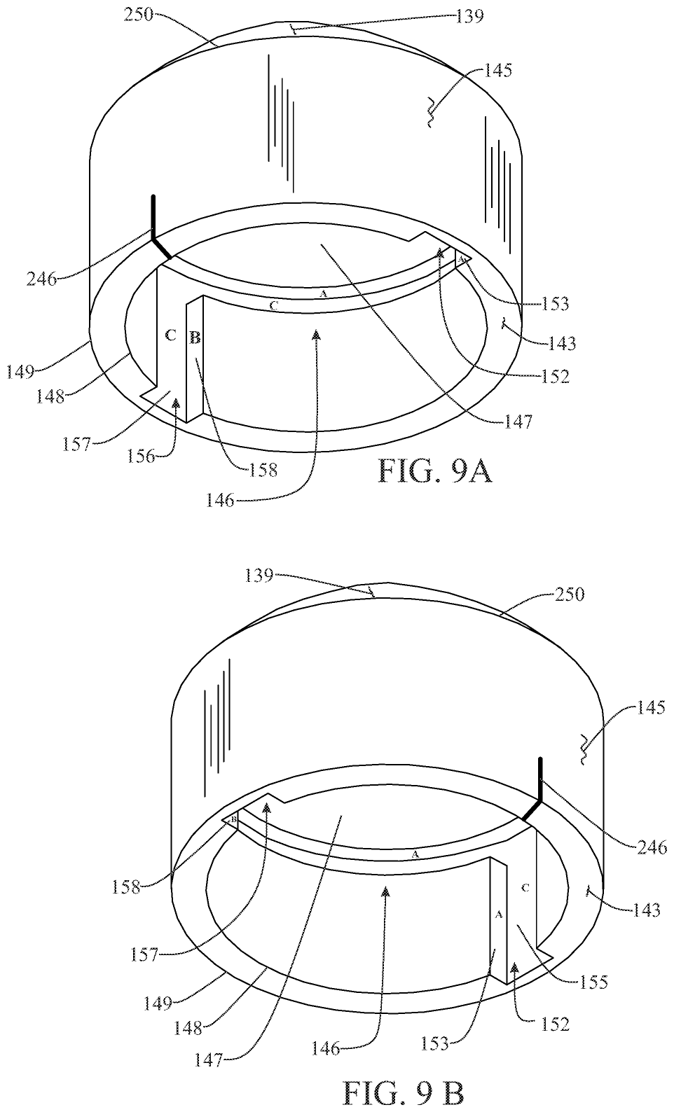

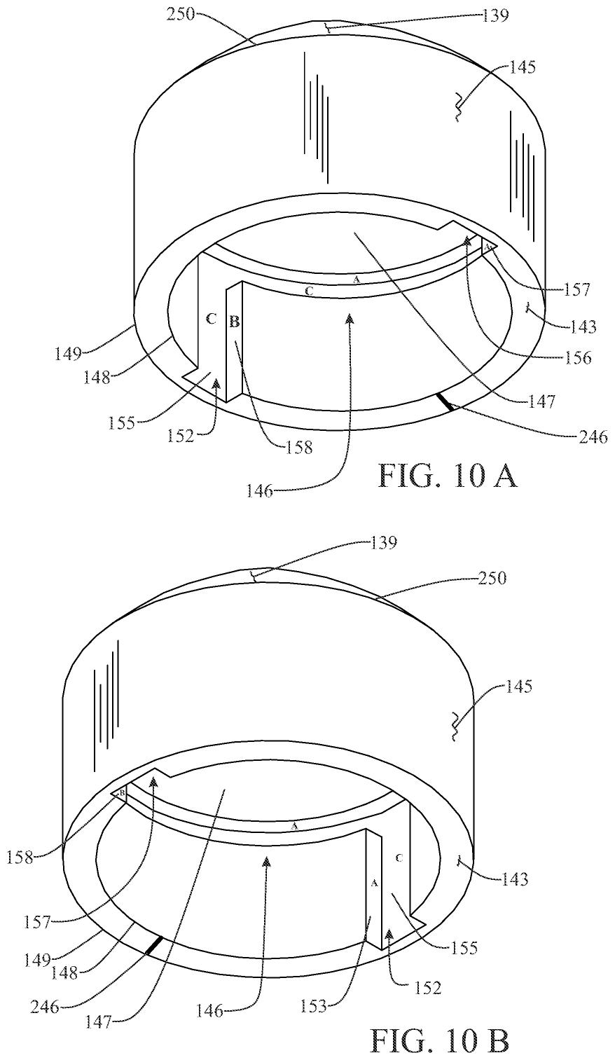

With reference to FIGS. 1-10B, a child resistant pocket medicinal substance container 100 generally includes a medicinal substance container lower end 116, a medicinal substance container middle section 117, a medicinal container upper end 118, a cylindrical medicinal substance container body 101, a domed medicinal substance container lid 102, a container power on & off button 255, a High Definition Camera 257, a first container Status Indicator LED Light 252, a second container status indicator LED light 253, a third container status indicator LED Light 254, a micro speaker 258 for playing music, a two dimensional magnetic nib sliding channel system 151, a medicinal substance container divider ring 103, a container orientation status marker 248, an electromagnetic lock system 600 with an electronic lock 601, a sterile refillable medicinal substance cartridge assembly 119 that includes a first sterile refillable medical substance cartridge 120 and a second sterile refillable substance cartridge 121 which can be retained within the cylindrical medicinal substance container body 101 (See FIGS. 4A-6B). The medicinal substance container divider ring 103 is located between the cylindrical medicinal substance container body 101 and the domed medicinal substance container lid 102 when the child resistant pocket medicinal substance container 100 is fully assembled and the domed medicinal substance container 102 is secured onto the cylindrical medicinal substance container body 101 (See FIGS. 8A & 8B). A portion or segment of the container orientation status marker 248 is found on all the parts of the lockable electronic pocket medicinal substance container 100 to help a user properly orient and assemble the child resistant pocket medicinal substance container 100 (See FIG. 5). When all its segments of the container orientation status marker 248 are aligned, the domed medicinal substance container lid 102 may be placed on or removed off the cylindrical medicinal substance container body 101. The container orientation status marker 248 also allows a user to know the locked or unlocked status of the child resistant pocket medicinal substance container 100 by giving the user a visual indicator to check (See FIGS. 8A & 8B). The positioning of the container orientation status marker 248 is strategically placed on predetermined status points. The container orientation status marker 248 may be two dimensional for example painted or printed on the child resistant pocket medicinal substance container 100 or it may be three dimensional at some sections so the status marker 248 can be felt like brail. The container power on & off button 25 is pushed and held for three seconds to turn on the child resistant pocket medicinal substance container 100. The micro speaker 258 can now be paired to a smart mobile computer (Not Shown) via a Bluetooth connection and play music stored on or streamed from the smart mobile computer.

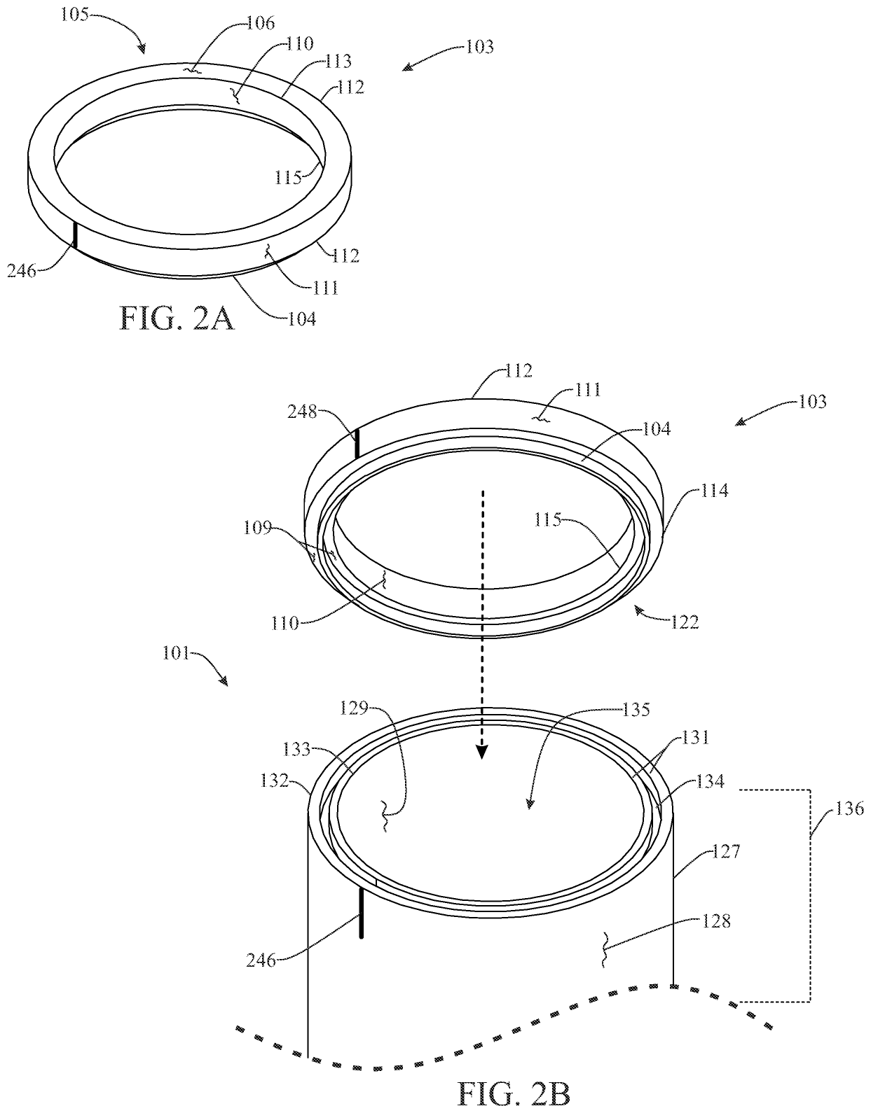

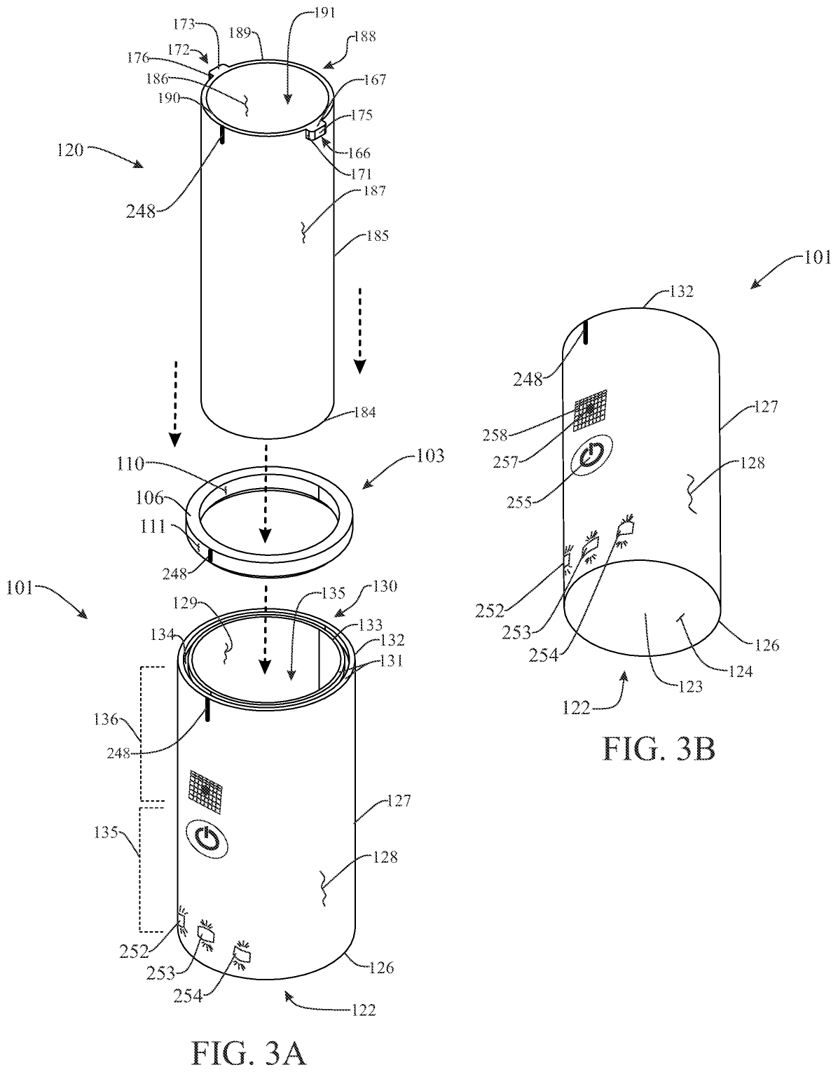

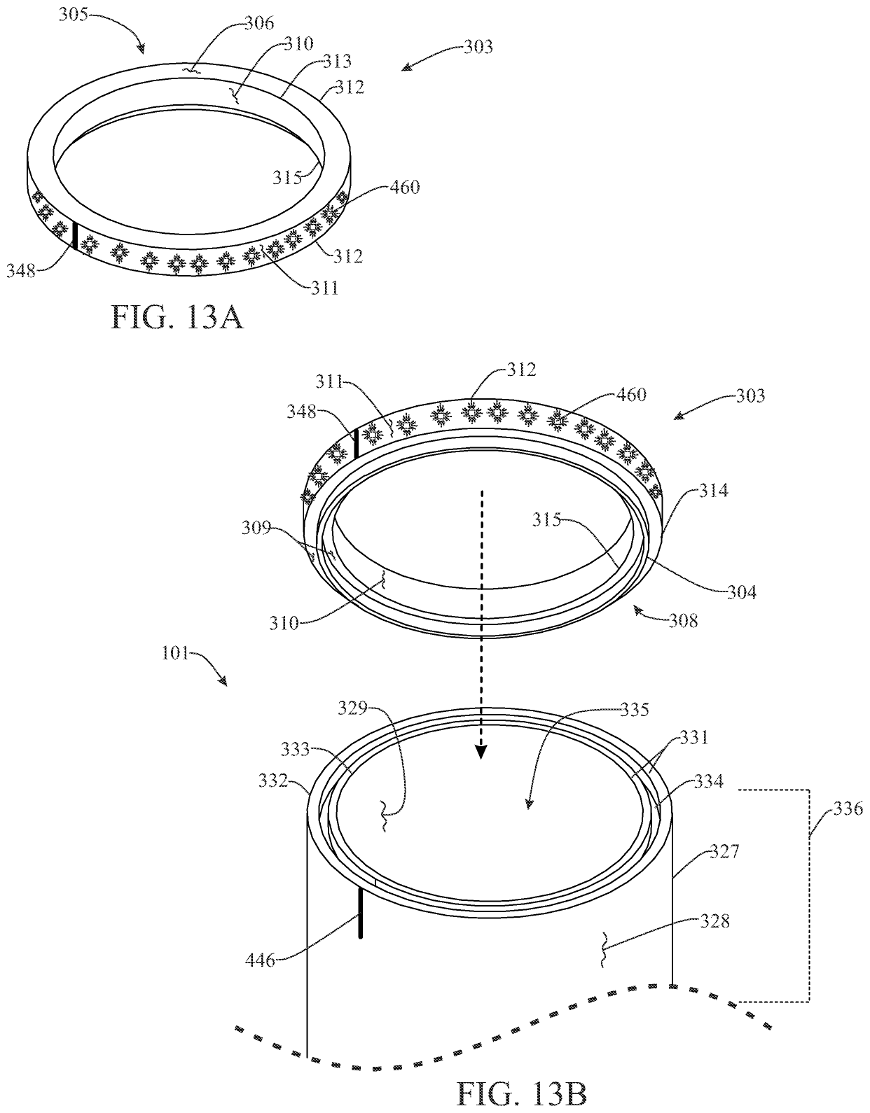

With reference to FIGS. 3A-2B, FIG. 5, FIG. 7B & FIG. 13B The cylindrical medicinal substance container body 101 generally includes a container body lower half 135, and a container body upper half 136, a circular container body base 122, a container body base center point 123, a container body base outer surface edge 126, a container body base inner surface cavity floor 125, a cylindrical container body wall 127, a container body wall top 130, a cylindrical container body wall top surface 131, a cylindrical container body cavity 135, and a cylindrical container body cavity entrance 530. The circular container body base 122 is located at the container body lower half 136 and includes the container body base surface center point 123, a circular container body base outer surface 122, and a circular container body base outer surface edge 124. The cylindrical container body wall 127 extends vertically from the circular container body base 122 at the circular container body base outer surface edge 126. The cylindrical container body wall 127 surrounds and towers the container body base inner surface cavity floor 125 defining the cylindrical container body cavity 135. The cylindrical container body wall top surface 131 is circular and surrounds the cylindrical container body cavity 135 defining a container body cavity entrance (Not Numbered) at the container body upper half 136. Cylindrical container body 101 may be fashioned with wood such as bamboo, mahogany, oak, pine, maple, or any other wood known to exist. The cylindrical medicinal substance container body 101 may be fashioned with plastic configured to look like wood by painting or other methods known to make plastic look and feel like wood. The cylindrical medicinal substance container body 101 may be made with plastic, or a material made from cannabis, hemp, or carbon fiber. The cylindrical medicinal substance container body 101 may be made with metal including precious metals such as gold, silver, platinum, copper, or any other metal known to man. The cylindrical medicinal substance container body 101 may be metal plated. The child resistant pocket medicinal substance container 100 may be decorated with precious stone such as diamonds, rubies, emeralds, sapphires, pearls, or any other precious stone known to exist. Cylindrical medicinal substance container body 101 may be decorated with fake stones or real crystals. As best shown in FIGS. 2A-3B, the cylindrical container body wall 127 includes the cylindrical container body wall top 130, the cylindrical container body wall top surface 131, a cylindrical container body wall outer surface 128 and a cylindrical container body wall inner surface 129, a cylindrical container body wall top surface outer edge 132 and a cylindrical container body wall top surface inner edge 133, and a cylindrical container body wall top surface circular divider ring lip groove 134. The cylindrical container body wall top circular divider ring lip groove 134 formed into the top surface 131 of the cylindrical container body wall 127.

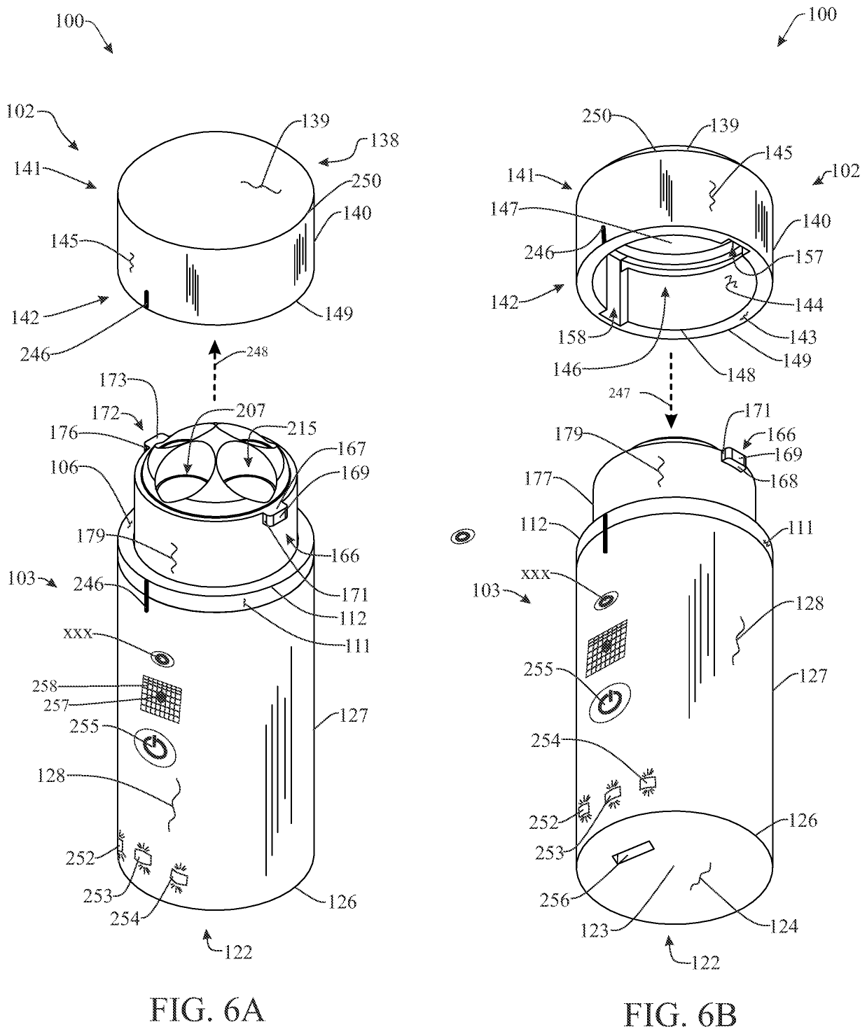

As best shown in FIGS. 2A & 2B, the medicinal substance container divider ring 103 includes a divider ring wall 107, a divider ring wall top 105, a divider ring wall base 108, a divider ring wall base surface 109, a divider ring wall outer surface 111, a divider ring wall inner surface 110, a divider ring wall top surface 106, a divider ring wall top outer edge 112, a divider ring wall top inner edge 113, a divider ring wall base outer edge 114, a divider ring wall base inner edge 115 and a divider ring support rail 104. The divider ring support rail 104 is circular and projects from the divider ring wall base 108. The divider ring support rail 104 is in the middle of and between the divider ring wall base outer edge 114 and the divider ring wall base inner edge 115. The divider ring support rail 104 is sized and shaped to snap fit in the cylindrical container body wall top circular divider ring lip groove 134 on the container body wall top surface 131 and be retained thereby. When the divider ring support rail 104 is fully inserted in the cylindrical container body wall top circular divider ring lip groove 134 (See FIG. 6A), the divider ring wall base surface 109 contacts with and sits on the container body wall top surface 131. The medicinal substance container divider ring 103 may be further secured to the body wall top surface 131 with an adhesive or snap fit. The medicinal substance container divider ring 103 may be fashioned with wood such as bamboo, mahogany, oak, pine, maple, or any other wood known to exist. The medicinal substance container divider ring 103 may be made with plastic to reduce cost, or carbon fiber to reduce weight and increase the container's strength, or hemp to help promote a green planet and reduce the effects of littered plastic and consumer waste. The medicinal substance container divider ring 103 may be made with metal including precious metal such as gold, silver, platinum, copper, or any other metal known to man.

The domed medicinal substance container lid 102 includes a container lid wall 140, a container lid wall top 141, a container lid wall bottom 142, a container lid wall bottom surface 143 a container wall bottom surface inner edge 148, a container wall bottom surface outer edge 149, a container lid wall inner surface 144, a container lid wall outer surface 145, a container lid roof 138, a container lid roof surface 139, a container lid roof surface edge 250, a container lid cavity 146, a container lid cavity ceiling 147, a two dimensional nib sliding channel 150, a first vertical nib channel portion 152, a second vertical nib channel portion 156, a circular nib channel portion 160, a first vertical nib channel surface A 153, a first vertical nib channel surface B 154, a first vertical nib channel surface C 155, a second vertical nib channel surface A 157, a second vertical nib channel surface B 158, a second vertical nib channel surface 159 C, a circular nib channel surface 161 A, a circular nib channel surface B 162, a circular nib channel surface C 163, a first nib channel entrance, 164, a second nib channel entrance 165, an electromagnetic lock System 600. As best shown in FIGS. 3A-3B, the lid roof 138 is dome shaped and is supported by the lid wall 140 at the container lid wall top 141. The container lid roof 138 covers the lid cavity 146 at the container lid wall top 141. The circumference of the lid wall outer surface 145 is equal to circumference of the lid roof surface edge 178 so at the container lid wall top 141, the container lid wall outer surface 145 ends at the lid roof surface edge 178 of the container lid roof 138. The container lid wall outer surface 145 is also equal to the circumference of the cylindrical container body wall outer surface 128 and the circumference of the divider ring wall outer surface 111 so the cylindrical medicinal substance container body 101 has a smooth outer surface. The container lid wall outer surface 145 ends at the container lid wall bottom surface outer edge 149 at the container lid wall bottom 142. The thickness of the container lid wall 140 is equal to the distance between the container lid wall bottom surface inner edge 148 and the container lid wall bottom surface outer edge 149. The container lid cavity 146 of the domed medicinal substance container lid 102 houses the two-dimensional nib sliding channel 150 of the two-dimensional nib sliding channel system 151. The two-dimensional nib sliding channel 150 includes a first and second vertical nib channel portion 152 & 156 and a circular nib channel portion 160. The first and second vertical nib channel portions 152 and 156 are carved out of the container lid wall bottom surface 143 and the container lid wall inner surface 144. The first and second vertical nib channel portions 152 and 156 extending vertically from the container lid wall bottom 142 up toward the container lid cavity ceiling 147. The first left vertical nib channel portions 152 and the second right vertical nib channel portion 156 provides the first nib channel entrance A 164 and the second nib channel entrance B 165 located at the container lid wall bottom surface 143. The plane of the first vertical nib channel surface A 153 and the plane of the first vertical nib channel surface B 154 are parallel to each other and perpendicular to the plane of first vertical nib channel surface C 155 of first left vertical nib channel Portion 152. Likewise, the plane of the second vertical nib channel surface A 157 and the second vertical nib channel surface B 158 are parallel to each other and perpendicular to the plane of second vertical nib channel surface C 159 of second right vertical nib channel Portion 156. The two vertical nib channel portions 152 and 156 are equal in size and shape and are opposite each other within the container lid cavity 146. The first and second vertical nib channel portions 152 and 156 stretch vertically until the six vertical nib channel surfaces 153, 154, 155, 157, 158, and 159 end where they meet a plane defined by the circular nib channel surface B 162. The first and second vertical nib channel portions 152 and 156 are perpendicular to the plane defined by the circular nib channel surface B 162. The circular nib channel portion 160 which is carved into the container lid inner wall surface 144 is located just below the edge of the ceiling of the lid cavity 147 at the container lid wall top 141. Note: The domed medicinal substance container lid 102 may be fashioned with totally transparent material glass or plastic or may be partially transparent. The domed medicinal container lid 102 may be configured in different sizes and shapes. The domed medicinal container lid 102 may be manufactured with different colors that can be coordinated to indicate different purposes, to identify a particular medicinal substance's classification in the container. The domed medicinal container lid 102 may double as a cannabis bud display cup, container, or bowl. These cups may be displayed in a lockable product display case traditionally used by jewelry retailers and medical cannabis dispensaries (Not Shown). These Lids (Not Shown) may be configured with a two-dimensional nib sliding channel like the container lid cavity 146 of the domed medicinal container lid 102 that houses the two-dimensional nib sliding channel 150 described.

The first and second vertical nib channels portions 152 and 156 are equal in size and shape and are opposite each other. The distance between the first and second vertical nib channel portions 152 and 156 is equal to the diameter of the container lid wall bottom surface inner edge 148. The container lid wall bottom surface 143 provides the first and second nib channel slide entrance 164 and 165 at the container lid wall bottom 142. The plane of the first vertical nib channel surface C 155 meets the planes of the first and second vertical nib channel surface A 153 and B 154 at a 90-degree angle creating the first vertical nib channel 152. The plane of the second vertical nib channel surface C 155 meets the planes of the second vertical nib channel surface A 157 and B 158, at a 90-degree angle creating the second vertical nib channel portion 156. The second vertical nib channel surfaces A 157 and B 158 are opposite and parallel each other. The electromagnetic lock 600 is housed in the circular nib channel portion 160. All the surfaces of the two-dimensional nib sliding channel system 151 may be magnetized. All the surfaces may be formed with circular shaped, crescent shaped, ring shaped or bar shaped magnets to operate the electromagnetic lock system 601.

The second sterile refillable medicinal substance cartridge 121 is sized, shaped, and designed to be inserted into the first sterile refillable medicinal substance cartridge 120 and be retained thereby. The second sterile refillable medicinal substance cartridge 121 includes a second refillable medicinal substance cartridge head 198, a first cylindrical substance chamber/tube 194, a second cylindrical substance chamber/tube 195, a third cylindrical substance chamber/tube 196, a fourth cylindrical substance chamber/tube 197 configured to and extending from the refillable medicinal substance cartridge head 198. The cylindrical substance chambers/tubes may be equal in shape and size.

The second refillable medicinal substance cartridge head 198 may be configured with sensors (Not Shown), a microcontroller or processor (Not Shown), LED Light/Lights (Not Shown), a camera in or on it (Not Shown). The second refillable medicinal substance cartridge head 198 has a second refillable medicinal substance cartridge head domed top surface 240, a second refillable medicinal substance cartridge head bottom flat surface 241, a second refillable medicinal substance cartridge head curved side surface 243, a second refillable medicinal substance cartridge head center point 242, a second refillable medicinal substance cartridge head first head hole 206, a second refillable medicinal substance cartridge head second head hole 215, a second refillable medicinal substance cartridge head third head hole 225, a second refillable medicinal substance cartridge head fourth head hole 235. The four head holes 206, 215, 225, and 235 respectively have a head hole inner surface, a first head hole inner surface 207, a second head hole inner surface 216, a third head hole inner surface 225 and a fourth head hole inner surface 233. The four head holes 206, 215, 225, and 235 are the respective entrances to each of the four cylindrical substance chambers/tubes 194, 195, 196, and 197. The four head holes 206, 215, 225, and 235 may be numbered or have another identifying marker (Not Shown). The head holes bore straight through the second sterile refillable medicinal cartridge head 121. The medicinal substances placed in the second sterile refillable medicinal substance cartridge 121 enter through the cartridge head holes 206, 215, 225, and 235. The head holes may be sized and shaped equal to the cylindrical substance chambers/tubes 194, 195, 196, and 197. The four head holes 206, 215, 225, and 235 and their respective entrances to each of the four cylindrical substance chambers/tubes 194, 195, 196, and 197 may be configured with sensors (Not Shown), cameras (Not Shown), retractable cavity doors (Not Shown) in or on each of the four head holes 206, 215, 225, and 235. The four head holes 206, 215, 225, and 235 may be the respective entrances to each of the four cylindrical substance chambers 194, 195, 196, and 197 and may be sealed with any Child-Resistant Seal or Method Disclosed anywhere herein. There may be more than four head-holes and chambers/tubes. For example, there may be 6, 8, 10 or more holes in the second refillable medicinal substance cartridge head 198. The holes may be aligned and arranged symmetrically and equally spaced around a center point of the second refillable medicinal substance cartridge head 198. The first cylindrical substance chamber 206 has a first chamber base 199, a first chamber base edge 202, a first chamber wall 203, a first chamber wall outer surface 205, a first chamber wall inner surface 204, a first chamber base inner surface floor 201, a first chamber opening or entrance 531 (Not Shown).

The first chamber base 199 may be configured to be removed or detached from the first cylindrical substance chamber 206 for cleaning. The removable may be realized by unscrewing the first chamber base 199 from substance chamber 196. The first chamber base 199 and the cylindrical substance chamber 206 may be outfitted with threads (Not Shown) so they can screw to each other. The ability for the first chamber base 199 to separate from the cylindrical substance chamber 206 is crucial to clean the first chamber base 199.

The second cylindrical substance chamber 196 has a second chamber base 208, a second chamber base edge 211, a second chamber wall 212, a second chamber wall outer surface 214, a second chamber wall inner surface 213, a second chamber base inner surface floor 210, a second chamber opening or entrance 532. The second chamber base 196 may be configured to be removed or detached from the first cylindrical substance chamber 208 for cleaning. The removing the second chamber base 196 may be realized by unscrewing the first chamber base 208 from the substance chamber 196. The second chamber base 208 and the cylindrical substance chamber 196 may be outfitted with threads (Not Shown) so they can screw to each other. The ability for the second chamber base 199 to separate from the second cylindrical substance chamber 196 is crucial for clean the first chamber base 199.

The third cylindrical substance chamber 196 has a third chamber base 218, a third chamber base edge 221, a third chamber wall 222, a third chamber wall outer surface 224, a third chamber wall inner surface 223, a third chamber base inner surface floor 220, a third chamber opening or entrance 533 (Not Shown). The third chamber base 218 may be configured to be removed or detached from the third cylindrical substance chamber 196 for cleaning. The removable may be realized by unscrewing the third chamber base 208 from the third cylindrical substance chamber 196. The third chamber base 218 and the third cylindrical substance chamber 196 may be outfitted with threads so they can screw to each other. The ability for the third chamber base 218 to separate from the third cylindrical substance chamber 218 is crucial for clean the first chamber base 199.

The fourth cylindrical substance chamber 197 has a fourth chamber base 228, a fourth chamber base edge 231, a fourth chamber wall 232, a fourth chamber wall 232, a fourth chamber wall inner surface 233, a fourth chamber base inner surface floor 230, a fourth chamber opening or entrance 534 (Not Shown). The fourth chamber base 197 may be configured to be removed or detached from the fourth cylindrical substance chamber 197 for cleaning. The removable may be realized by unscrewing the fourth chamber base 197 from the fourth cylindrical substance chamber 197. The fourth chamber base 228 and the fourth cylindrical substance chamber 197 may be outfitted with threads so they can screw to each other. The ability for the fourth chamber base 228 to separate from the fourth cylindrical substance chamber 197 is crucial for cleaning the fourth chamber base 228.

The first medicinal substance cartridge head hole 206, the second the cartridge head hole 215, the third cartridge head hole 224, and the fourth cartridge head holes 235 lead to the four cartridge chamber/tube entrances 531, 532, 533, & 534 (Not Shown) located in the cartridge head 198 when the cylindrical substance chambers/tubes 194, 195, 196, and 197 are coupled or screwed into the cartridge head 198. The second refillable medicinal substance cartridge head 198 is configured to connect/couple or screw into the second refillable medicinal substance cartridge head bottom flat surface 241 to the top of the four cylindrical substance chambers 194, 195, 196, and 197. The second refillable medicinal substance cartridge head 198 is configured to be attached in a manner where the four chamber openings (Not Shown) are aligned with the four cartridge head holes 206, 215, 225, and 235. Any medicinal substance placed in the second sterile refillable medicinal substance cartridge 121 may enter through the cartridge head holes 206, 215, 225, and 235 to be sealed with any of the child resistant methods disclosed herein. For example, a child resistant cover seal (Not Shown) may be used for sealing the four cartridge chamber/tube entrances 531, 532, 533, & 534 of the four cylindrical substance chambers/tubes 194, 195, 196, and 197. The cover seal may be the type that is peeled off, screwed off, or pierced through allowing access into the chambers/tubes 194,195,196, and 197. The head holes/ports 206, 215, 225, and 235 may have dimensions and shapes equal to the four cylindrical substance chambers 194, 195, 196, and 197. The second refillable medicinal substance cartridge head 198 and the four cylindrical substance chambers 194, 195, 196, and 197 may be formulated as one unit or configured to connect/couple to each other via a snap in fit method or screwing into fastener holes bored into the cartridge head bottom surface 241. Once the cylindrical substance chambers/tubes 194, 195, 196, 197 are screwed or secured to the second refillable medicinal substance cartridge head 198, the four base surfaces 200, 192, 219, and 228 of the cylindrical substance chambers 194, 195, 196, and 197 are placed within the circumference of the first medicinal substance cartridge cavity top inner edge 190. The second sterile refillable medicinal substance cartridge 121 is then pushed and slid into the first refillable medicinal cartridge cavity 191 to be retained thereby (See FIGS. 4A & 4B). Once the second sterile refillable medicinal substance cartridge 121 is fully inserted into first sterile refillable medicinal substance cartridge 120, the domed medicinal substance container lid 102 may be placed on (See FIGS. 6A & 6B, 8A & 8B). (Note: The Second sterile refillable substance cartridge 121 may be removed and the user may elect to use the child resistant pocket medicinal substance container 100 with just the first sterile refillable medicinal substance cartridge 120 or vice versa. The cavity of the first sterile refillable medicinal substance container 191 has more volume or space and can contain other shaped capsules, containers, or cartridges. For example, a cannabinoid substance jar (Not Shown) can be placed into the cavity of the first sterile refillable medicinal substance cartridge 191. The cannabinoid substance in the cannabinoid substance jar may be a cannabinoid wax, a cannabinoid butter, a cannabinoid oil, a cannabis flower, a cannabis infused eatable candy, or any other cannabis or cannabinoid infused product. The first sterile refillable medicinal substance cartridge 120 also can hold a larger capacity of cannabis cigarettes or cannabis cigars then the second refillable substance cartridge. When the first sterile refillable medicinal substance container 120 has a cannabinoid substance jar (Not Shown) filled with cannabis oil, butter, wax or any another cannabis infused product, the second refillable substance cartridge may be sized and shaped to be inserted into the first sterile refillable medicinal substance cartridge 120 to rest upon the cannabinoid substance jar lid top. The cannabinoid substance jar may be like those used in lip balm packaging where the user dips their finger in plastic or small glass jar or container to apply the lip balm to their finger and then lips. The cannabinoid substance jar or small containers having lids that screw on and off and are sized and shaped to be received by the medicinal substance container body 101 or the first sterile refillable medicinal substance cartridge 120.

As best shown in FIGS. 6A & 6B, 8A & 8B, 9A & 9B and 10A & 10B, once the second sterile medicinal substance cartridge 121 and the first sterile refillable medicinal substance cartridge 120 are fully engaged and inserted in the medicinal substance container body 101, the domed medicinal substance container lid 102 may be secured to the body. A first left side nib key front surface 169 contacts the first vertical nib channel surface C 155, a first left nib key right side surface 171 contacts the first vertical nib channel surface B 154 and a first nib key left side surface 170 contacts the second vertical nib channel surface A 153. Once inserted, a second nib key front surface 175 contacts the second vertical nib channel surface C 159, a second nib key right side surface 177 contacts the second vertical nib channel surface B 158 and a first nib key left side surface 176 contacts the second vertical nib channel surface A 157. The respective surfaces of the nib keys 166 and 172 contact the respective surfaces of the first and second vertical nib channel portion 152 and 156 in a way that enables the first and second nib keys 166 and 172 to slide on an up the first and second vertical nib channel portion 152 and 156. The diameter of the first refillable medicinal container cartridge top outer edge 189 is slightly smaller than the diameter of the container lid wall bottom surface inner edge 148 enabling the first refillable medicinal container cartridge wall top 188 and a portion of the first refillable medicinal substance cartridge wall outer surface 187 to slide up into the container lid cavity 146 when the Nib keys 166 and 172 are aligned with the vertical nib channel portions 152 and 156. The top of the first refillable medicinal substance cartridge wall outer surface 187 then engages with and slides against the container lid inner wall inner surface 144. The first and second nib keys 166 and 172 slide up until the first and second nib key top surfaces 167 and 173 cross the plane defined by the circular nib channel surface B 162 into the circular nib channel portion 160 and contact the circular nib channel surface A 161 where nib keys 166 and 172 are stopped from sliding vertically any further. Once the nib keys top surfaces 167 and 173 contact the circular nib channel surface A 161, and the container lid wall bottom surface 143 contacts the divider ring wall top surface 106, the domed medicinal substance container lid 102 can be turned clockwise or counterclockwise engaging the first and second nib key bottom surfaces 167 and 174 with the circular nib channel surface B 162 (See FIGS. 8A-8B). The circular nib channel portion 160 designed to slide on and around the nib keys 166 and 172. Once the container lid is rotated enough to where the nib keys 366 and 372 are un aligned with the first and second vertical nib channel portions 152 and 156, the container lid 102 is secured or locked onto the container body 101. A user can than turn the container lid 102 rotating the circular nib channel portion 360 around the first and second nib keys 166 and 172 around the circular nib channel 360 until the first and second nib keys are aligned once again with the vertical nib channel portions 152 and 156. Once aligned, the user can pull the container lid 102 upward and removed it thereby opening the child resistant medicinal substance container 100.

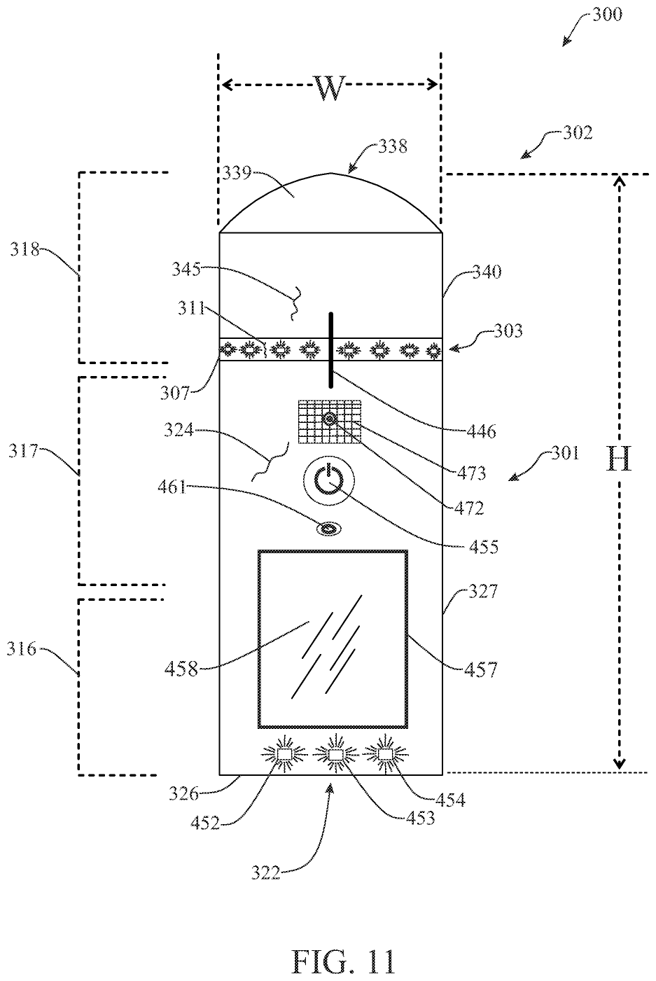

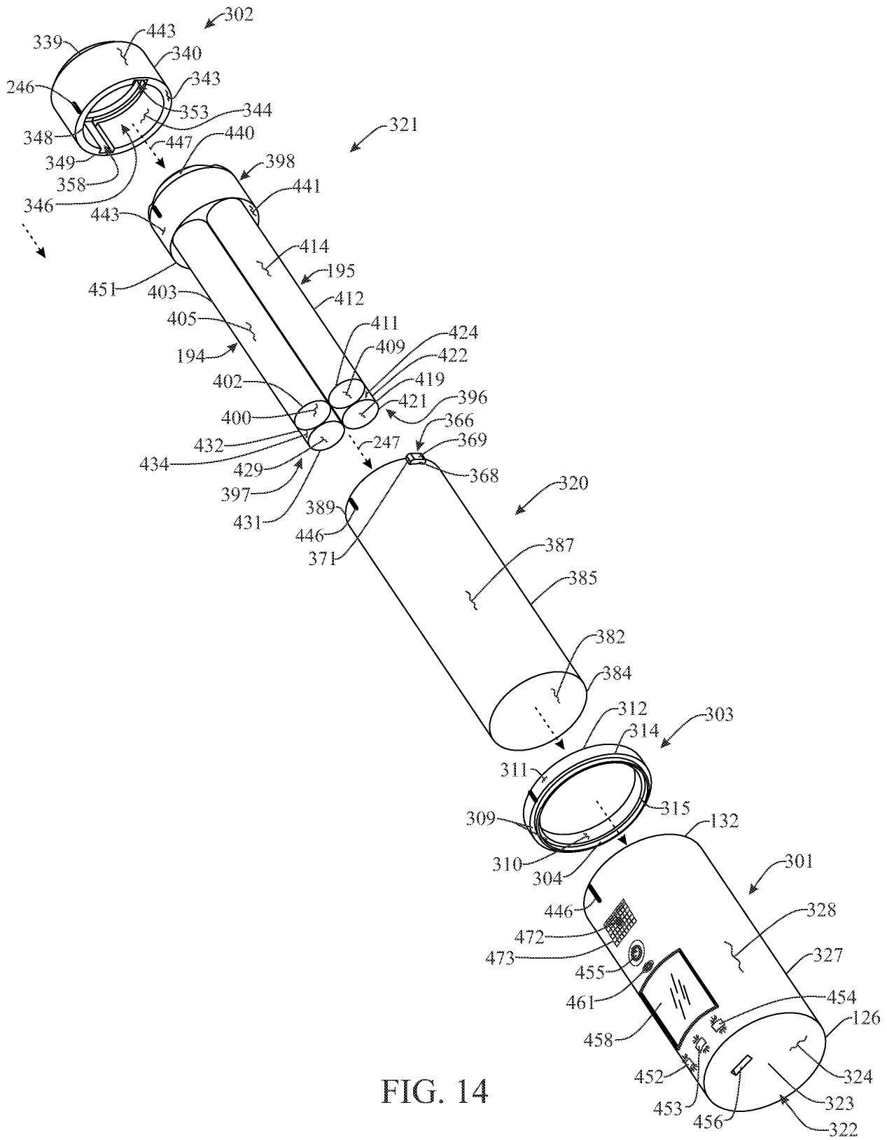

Referring now to FIGS. 11-15 there is disclosed an alternative embodiment of a child resistant medicinal substance container 300 being a part of a system designed for use in authenticating, conveying, securing, and tracking a first sterile refillable substance cartridge 320 filled with a prescribed cannabis flower, or a prescribed cannabinoid medicinal substance. With reference to FIGS. 11 & 12, the child resistant medicinal substance container 300 generally includes a medicinal substance container lower end 316, a medicinal substance container middle section 317, a medicinal container upper end 318, a cylindrical medicinal substance container body 301, a domed medicinal substance container lid 302 with a two dimensional nib sliding channel system 352, a medicinal substance container magnetic divider ring 303, a container orientation status marker 446, a user interface 457, a microphone 463, micro-speaker 472, a high definition digital camera 461, a square touchscreen 458, a power/on button 245, divider ring programmable LED lights 460, a Blue Power On Light 452, a Red Battery Low Light 453, a Green Connected to Network/Bluetooth or Peripheral Smart Device Light 454, a USB Type C Data/Charging Port 456, a GPS module 463, a wireless interface in the form of a Bluetooth Module 474, a smart container software 471 internal memory device 467, computer processor 468, a digital scale 479. The child resistant medicinal substance container 300 also includes a refillable medicinal substance cartridge assembly 319 that includes a first sterile refillable substance cartridge 320 and a second sterile refillable substance cartridge 321 designed and shaped to be retained within the cylindrical medicinal substance container body 301. The second sterile refillable substance cartridge 321 may have medicinal substance cartridge head 398, and four cannabis medicinal substance chambers/tubes 394, 395, 396, & 397. The medicinal substance container magnetic divider ring 303 is located between the cylindrical medicinal substance container body 301 and the medicinal substance container lid 302 when the handheld the child resistant medicinal substance container 300 is fully assembled and the domed medicinal substance container lid 302 is on. A portion of container orientation status marker 446 may be located on all the parts of the child resistant medicinal substance container 300. The container orientation status marker 446 is designed to help a person properly orient and assemble the elements of the child resistant medicinal substance container 300. When all its elements are aligned the medicinal substance container lid 302 can be placed on or removed off the medicinal substance container body 301. The container orientation status marker 446 also allows a user to know the locked or unlocked status of the child resistant medicinal substance container 300 by giving the user a visual indicator to check. The positioning of the container orientation status marker 446 is strategically placed for predetermined status points. The container orientation status marker 446 may be two dimensional, for example, it may painted or printed on the child resistant medicinal substance container 300 or it may be three dimensional so that the status marker 348, can be felt like brail. A portion of the status may be marked on every element that makes up the child resistant medicinal substance container 300. The child resistant medicinal substance container 300 may send and receive data to and from a Customized Smart Container Centralized Computer Network 466 via a communication link 485. The communication link may be Wi-Fi.

The user interface 457 may be located on the medicinal substance container body 301 on the front of the child resistant medicinal substance container 300 and may be configured with a touchscreen 458. Although the exemplary image shows the user interface 457 configured to and located on cylindrical container body wall 327 it is understood the interface 457 may be configured to the container lid 302. The touchscreen 458 may be curved to rest flush or level with the curved cylindrical container body wall outer surface 328. The touchscreen 458 may be of the Resistive, Surface Acoustic wave, or Capacitive Touchscreen types. Touchscreen 458 may have the dimensions of 39 mm×45.2 mm or smaller. The touchscreen 458 may have a resolution of 368×448 pixels at 326 Pixels Per Inch. The Touchscreen 458 may be programed to record and recognize a user's touch patterns, movement patterns, and finger pressure sensitivity. The child resistant medicinal substance container 300 may a smart container software 471. The smart container software 471 may include a calendar app, a camera app, a voice recorder app, a Google app, a barcode and label scanner app, a custom light controlling app made specifically for the child resistant medicinal substance container 300, a google chrome browser app, a digital scale app, a cannabis dispensary product menu app, or any medical cannabis themed user app. The touchscreen 458 may be of the Resistive, Surface Acoustic wave, or Capacitive Touchscreen types. Touchscreen 458 may have the dimensions of 39 mm×45.2 mm or smaller. The touchscreen 458 may have a resolution of 368×448 pixels at 326 Pixels Per Inch. The Touchscreen 458 may be programed with a software that may record and recognize a user's touch patterns, movement patterns, and finger pressure sensitivity.

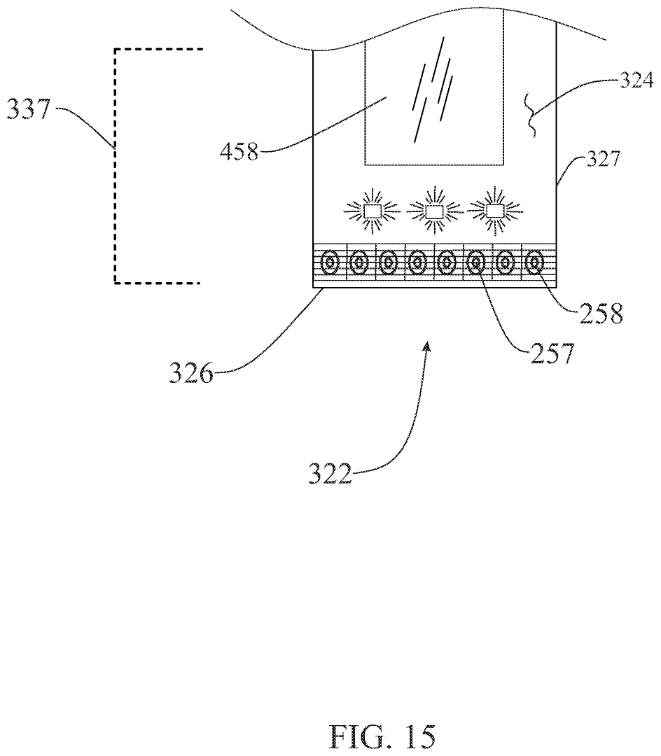

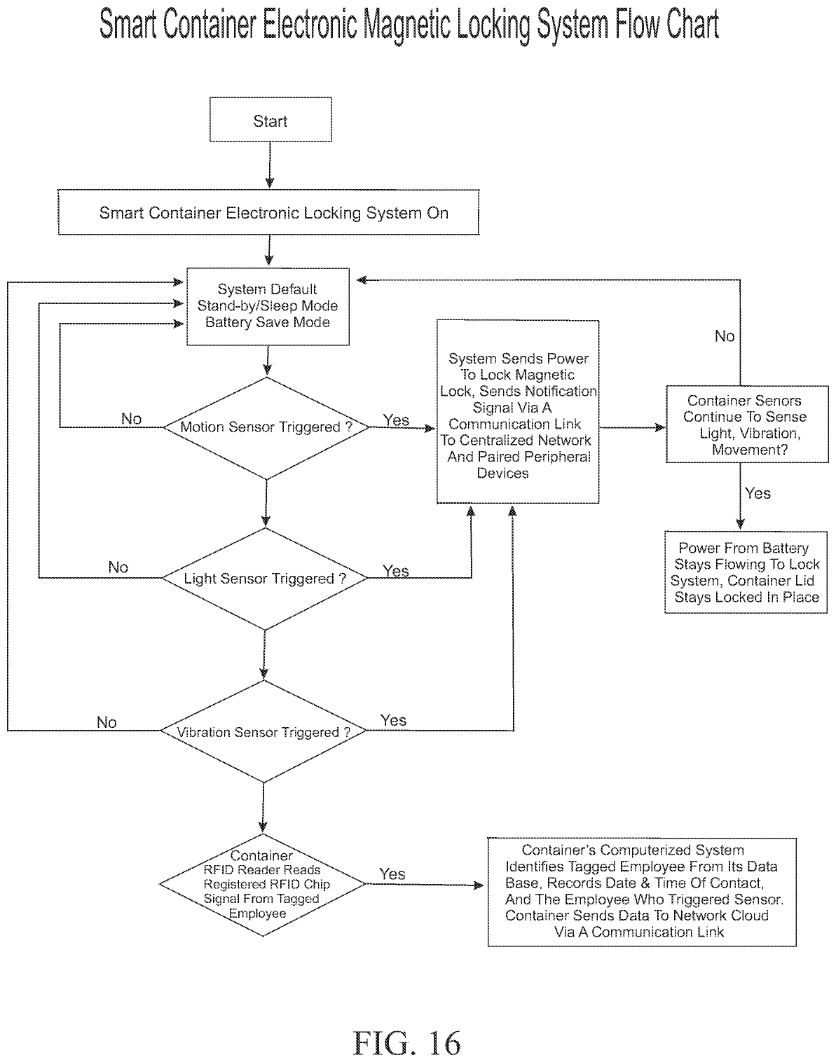

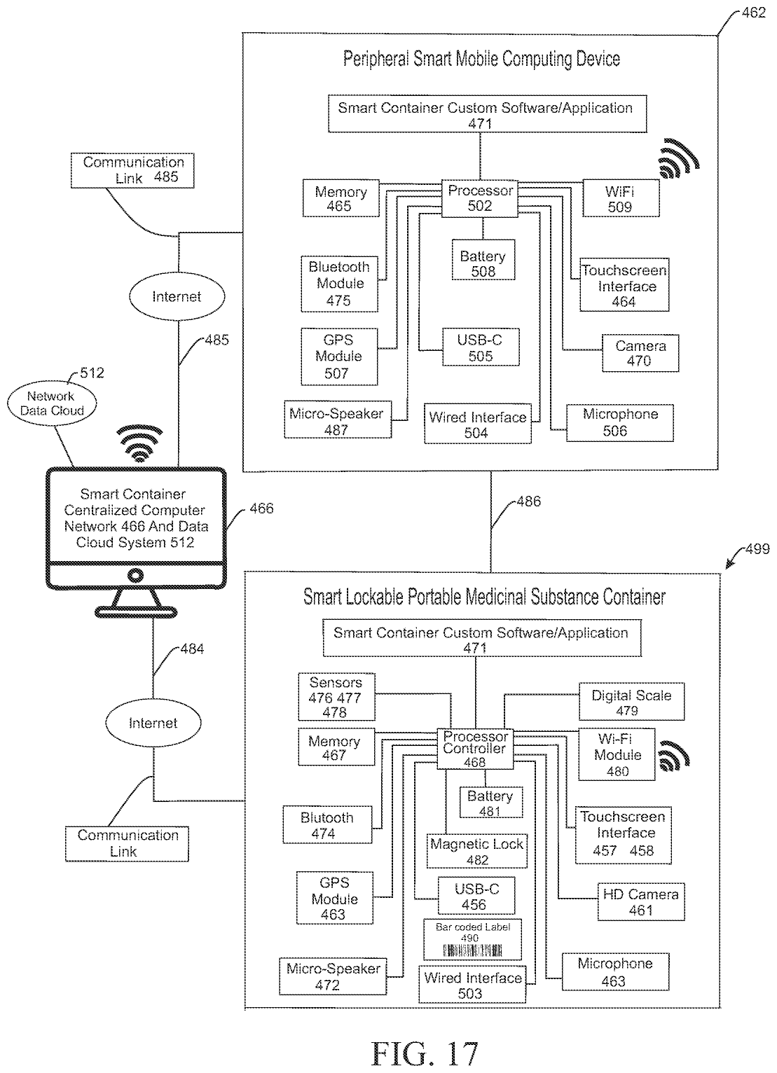

The child resistant medicinal substance container 300 may have a micro speaker 472 and a micro speaker grill 473. The micro speaker 472 may be a speaker small enough to fit into a cavity with the dimensions of 5 mm×24.4 mm×24.4 mm, The micro speaker 472 may be flat and be any micro speaker having at least one dimension being any measurement between the sizes of 4 mm 20 mm. The Micro speaker grill 473 as shown in FIG. 11 is configured from elongated vertical and horizontal bars. It is noted that Micro speaker grill 473 may be perforated with many small circular holes or with any other shape grill as long as it protects the micro speaker 473. As best shown in FIG. 15, there is another configuration where the child resistant medicinal substance container 300 features multiple micro speakers 257. The multiple micro speaker 257 may be configured to be placed on or in the cylindrical container body wall outer surface 327 at the cylindrical container body wall lower half 337. The multiple micro-speakers may be placed in a horizontal position that is parallel with a plane defined by the circular container body base outer surface 324. With this alternative configuration the Micro Speaker grill 473 may be located just above the circular container body base outer surface edge 326 on the cylindrical container body wall outer surface 328 at the cylindrical container body lower half 337. The Micro Speaker/Speakers 472 and the Micro Speaker/Speakers grill 473 may go around the perimeter of the cylindrical container body wall outer surface 328 at the cylindrical container body lower half 337. The micro speaker 472 in this alternate configuration may be configured to fit in a horizontal orientated cavity with the dimensions of 15 mm Wide×5 mm High×15 mm deep that can fit (NOTE) The micro speaker grill 472 may be snapped in a hole or cavity on the surface of the cylindrical body wall outer surface 328 or be formed in or with the cylindrical container body wall 327 so they are one unit as long as they allow the sound coming from the Micro Speaker 472 or speakers to project clearly and travel out so they may be heard clearly by a person or patient. By positioning the micro speakers 472 at the bottom, the sound projected from the speakers can travel across the surface that the child resistant medicinal substance container 300 sits on. (NOTE) The micro speaker 472 of the child resistant medicinal substance container 300 may play music from a Bluetooth signal sent from a Bluetooth Module 475 of the “Paired” peripheral smart portable computing device 462. The peripheral smart computing device touchscreen 458 of the Bluetooth paired peripheral smart portable computing device 462 may control the volume of the Micro speaker 472. The side positioned volume buttons of the peripheral smart portable computing device 462 may also control the volume of the Micro speaker 472. The peripheral smart computing device touchscreen 458 user interface may also download a firmware or the Smart Container Custom Software/Application 471 and be able to control all functions and elements of any variation or embodiment of the child resistant medicinal substance containers disclosed herein including but not limited to all of the LED Light/Lights 460 and their systems features disclosed herein, control and manipulate all features of all the remote controlled electromagnetic locking system 600 disclosed herein, a control the micro speakers 472, receive and manipulate all GPS functions disclosed herein, and control a High-definition cameras 461.

The child resistant medicinal substance container 300 may use the high-definition digital camera 461 to record anyone in its vicinity. The high-definition camera 461 may be configured to be controlled via a touchscreen 464 of a peripheral smart portable computing device 462 (Smartphone-Smartwatch-Tablet-Laptop-Desktop-Smartwatch) or the user interface/touchscreen 457 & 458 of the child resistant medicinal substance container 300. The high-definition camera 461 may have a camera lens (not shown). The touchscreen 464 may have camera zoom, record/stop record, and any other controls known in the art to manipulate the high-definition camera 461. The high-definition camera 461 may be configured to be utilized with facial recognition software that is installed in the internal memory 467 and executed by computer processor 468. The child resistant medicinal substance container 300, via the high-definition digital camera 461, may scan and record an image of the users face and store the image data onto its internal memory 467. The child resistant medicinal substance container 300 may scan, record, and recognize the users face via the high-definition camera 461. and unlock the child resistant medicinal substance container 300 electromagnetic locking magnetic locking system 489 if the face is authorized. The High-definition camera 461 may give a person the ability to unlock the touchscreen 457 and get into the Medicinal substance monitoring system 499 without having to enter a password/passcode. The high-definition camera 461 may also record hand gestures/movements or other body part gesture/movement and save it to an internal memory 467 for later person recognition. The image data may also be saved in memory 465 of the peripheral smart portable computing device 462 that is “paired” with the child resistant medicinal substance container 300 and is connected to the centralized network 466. (Note) A facial, a retina, a fingerprint, or any other Biometric Authentication Device System of the peripheral smart portable computing device 461 may be used to unlock all systems and features of the smart medicinal substance container 300 when the peripheral computing device 462 is “link/paired” via a communication link 486 to the child resistant medicinal substance container 300 and has access to the network 466 and the Smart Container Custom Software/Application 471 installed on/in. The peripheral smart portable computing device 462 may use its touchscreen 464, high-definition camera 470, scanner app, or any other smart mobile computing device feature known in the art to authenticate biometric data and allow access to locked and password secure encrypted systems. (Note) The touchscreen 458 may be used as the fingerprint scanner or an independent fingerprint scanner (Not Shown) may be configured to the child resistant medicinal substance container 300 on the cylindrical medicinal substance container body 301 or container lid 302. The high-definition camera 461 may be configured to aim up and down using a camera pivoting feature (Not Shown).

Referring to FIGS. 11 & 12 & 13B, the cylindrical medicinal substance container body 301 generally includes the container body lower half 316, the container body mid-section 317, and the container body upper half 318, a circular container body base 322, a container body base center point 323, a container body base outer surface edge 326, a container body base inner surface cavity floor 325, a cylindrical container body wall 327, a container body wall top 330, a cylindrical container body wall top surface 331, a cylindrical container body cavity 335, and a cylindrical container body cavity entrance 530. The circular container body base 322 is located at the container body lower half 336 and includes a container body base surface center point 323, a circular container body base outer surface 322, and a circular container body base outer surface edge 324. The cylindrical container body wall 327 extends vertically from the circular container body base 322 at the circular container body base outer surface edge 326. The cylindrical container body wall 327 surrounds and towers the container body base inner surface cavity floor 325 defining the cylindrical container body cavity 335. The cylindrical container body wall top surface 331 is circular and surrounds the cylindrical container body cavity 335 defining a container body cavity entrance (Not Numbered) at the container body upper half 336. The cylindrical container body 301 may be fashioned with wood such as bamboo, black walnut, mahogany, oak, pine, maple, or any other wood known to exist. The container body 301 may be fashioned with plastic configured to look like wood by painting the container body 301 or other methods known in the art to make plastic look and feel like wood. The container body 301 may be made with any plastic, a cannabis or hemp fashioned material, or carbon fiber. The cylindrical container body 301 may be made with metal including precious metals such as gold, silver, platinum, copper, or any other metal known to man. The cylindrical container body 301 may be precious metal plated. The child resistant medicinal substance container 300 may be decorated with precious stones such as diamonds, rubies, emeralds, sapphires, pearls, or any other precious stone known to exist. The cylindrical container body 301 may be decorated with fake stones or real crystals. (Note the divider ring 303 may have diamonds or any of the precious stones mentioned above set in the divider ring wall outer surface 311 of the divider ring 303. As best shown in FIG. 13B, the cylindrical container body wall 327 includes the cylindrical container body wall top 330, the cylindrical container body wall top surface 331, the cylindrical container body wall outer surface 328 and the cylindrical container body wall inner surface 329, the cylindrical container body wall top surface outer edge 332 and the cylindrical container body wall top surface inner edge 333, and the cylindrical container body wall top surface circular divider ring lip groove 334. The cylindrical container body wall top circular divider ring lip groove 334 formed into the top surface 331 of the cylindrical container body wall 327. (Note) An electronic parts compartment may be designed into the smart container body (Not Shown). The electronic compartment may be designed to retain the electronic components of the computer such as the memory device 467, the microcontroller 468, the micro-speakers 472, the microphone 463, the high-definition camera 461, USB-C 456, or a smart container system motherboard (Not Shown). The Electronic Compartment may be located at the cylindrical container body lower half 316 between the circular body base outer surface 324 and the circular container body base inner surface cavity floor 325. Tunnels, (Not Shown) may be formed in the cylindrical container body wall 327. The tunnels may be used to run Medicinal substance monitoring system 499 electrical wires and or data signal wires (Not Shown). The tunnels may be positioned vertically, or they may run around in between the container body wall inner and outer surface 328 & 329. The container body wall 327 may be completely or partially hallow and may be designed to run wires, or to embed hardware including but not limited to modules, CPU, chips, sensors, scanners, cameras, or memory. The smart container system motherboard (Not Shown) may be configured to sit flat or horizontal in the electronic compartment. The smart container system motherboard (Not Shown) may also be configured to sit at a 45-degree or 90-degree angle. The container body wall top surface 331 may be configured to be metallic or magnetic using any of the methods disclosed herein for surfaces to be magnetic. The surfaces (Not Numbered) of cylindrical container body wall top circular divider ring lip groove 334 may also be configured to be metallic or magnetic to attract a magnetic or metallic divider ring 303. The container body wall top surface 331 and of cylindrical container body wall top circular divider ring lip groove 334 may be configured to supply power to the LED light/Lights 460 configured with the divider ring 303 via any method known in the art for a surface to supply power to another surface.

As best shown in FIGS. 13A & 13B, the medicinal substance divider ring 303 includes a divider ring wall 307, a divider ring wall top 306, a divider ring wall base 308, a divider ring wall base surface 309, a divider ring wall outer surface 311, a divider ring wall inner surface 310, a divider ring wall top surface 307, a divider ring wall top outer edge 312, a divider ring wall top inner edge 313, a divider ring wall base outer edge 314, a divider ring wall base inner edge 315 and a divider ring support rail 304. The divider ring support rail 304 is circular and projects from the divider ring wall base 308. The divider ring support rail 304 is in the middle of and between the divider ring wall base outer edge 314 and the divider ring wall base inner edge 315. The divider ring support rail 304 is sized and shaped to snap fit in the cylindrical container body wall top circular divider ring lip groove 334 on the container body wall top surface 331 and be retained thereby. When the divider ring support rail 304 is fully inserted in the cylindrical container body wall top circular divider ring lip groove 334, the divider ring wall base surface 309 contacts with and sits on the container body wall top surface 331. The divider ring 303 may be further secured to the body wall top surface 331 with an adhesive. The divider ring 303 may be fashioned with wood such as bamboo, mahogany, oak, pine, maple, or any other wood known to exist. The divider ring 303 may be made with plastic to reduce cost, carbon fiber to reduce weight and increase the container's strength, or hemp to help promote a green planet and reduce the effects of littered plastic and consumer waste. The divider ring 303 may be made with metal including precious metal such as gold, silver, platinum, copper, or any other metal known to man. The divider ring 303 may be magnetized or completely made from ring shaped magnet. A magnet divider ring 303 may be used to increase the strength of the electromagnetic locking system 600. The Divider ring 303 may be lined with colored LED lights 460. The Divider ring 303 may be made with a translucent material (Not Shown) such as glass or plastic with another set of LED lights configured behind, below or directly from inside of the divider ring 303. The LED lights 460 may shine through the divider ring giving a halo effect. The divider rings LED lights 460 may be configured to flash, or light up in sequence. For example, the LED lights 460 may be programed to run in sequence and emit light in timed patterns individually or in grouped lights in harmony. The light/lights 460 may emit light one after another chronologically in a certain direction traveling around the ring so illumination circles the ring like a glowing halo. Light colors may be red, orange, yellow, green, blue, purple, sky blue turquoise, purple, fluorescent colors, or any of various shades of the rainbow or light spectrum. The divider ring 303 may be removable or detachable so to allow a person to interchange the divider ring 303 for decoration or medicinal substance identification, classification, or potency purposes. For example, a blue ring may mean that a dangerous or addictive drug is contained in the container, while a red ring may mean that the substance in the container medicinal substance container is dangerous to be handle by pregnant woman. A purple lit ring may indicate a sativa cannabis Strain is being locked and contained in the child resistant medicinal substance container 300 or yellow colored ring or LED colored ring lights 460 may represent that an indica strain vs a sativa strain is locked in the child resistant medicinal substance container 300. (Note) The LED programmable lights 460 may be arranged in 2, 3, 4, 5, or 6 rowed sequences in vertical or horizontal lines. and configured to any element the child resistant medicinal substance container 300 including but not limited to the smart container lid 302, and the smart container body 301. The peripheral smart portable computing device 462 may have the Smart Container Custom Software Application 471 installed specifically designed to control all the features of the LED light/lights 460 disclosed above or herein. The peripheral smart portable computing device 462 may control the child resistant medicinal substance container 300 or/and all the variants of containers and the embodiments of the containers disclosed herein via a communication link 486. For example, a person may use the touchscreen 464 of the peripheral smart portable computing device 462 to send a signal to the processor 468 to send a command signal to the LED light/lights 460 to make them flash, change color, dance, flow, or run around the divider ring 303. The peripheral smart portable computing device 462 may control the LED lights 460 of the child resistant medicinal substance container 300 and dim or enhance the brightness of the smart medicinal substance container system light/lights 460 via the communications link 486.

The child resistant medicinal substance container 300 may be programed to change the LED lights 460 to an exclusive or authorized color/colors not accessible to any user. The exclusive color or colors may be chosen for example by a Brand Marketing Agency by a Licensed Medical Cannabis Cultivator, Distributer, or Dispensary. The elected color (“VIP Color”) and it's assigned pattern or light dance may be promoted and used to advertised and market the launch of a new cannabis product or strain. For example, a new patented and branded cannabis strain genetically modified by scientist and grown to increase the appetite of cancer patients may be assigned a sky blue or powder blue light color and a circular light pattern. The code to enable the LED lights 460 is written into the law compliant label 490. The new strain is then made authenticable by the LED lights 46o when the child resistant medicinal substance container 300 emits that sky blue “VIP Color”. For Example, a person may use the scan feature on the peripheral smart portable computing device 462 to scan the cannabis law compliant label 490 adhered to the sterile refillable medicinal substance cartridge 320. Once the cannabis law compliant label 490 is scanned and read by the scan feature of the peripheral smart portable computing device 462 the Once the centralized computer network system law compliant label 490 is scanned and passes the authentication test, the peripheral smart portable computing device user interface touchscreen 464 may display all the information stored to/on the law compliant label 490 of the strain contained in the sterile refillable medicinal substance cartridge 320. the sealed labeled refillable cartridge, tube, jar, container, bag etc. The information/data displayed on the touchscreen 464 may be any data that is disclosed herein that may be able to be listed on the label including but is not limited to seed information (genetics/strain), plant and harvest dates, plant strain name, plant “Lab” results, harvest growers license numbers, state issued logo, THC, CBD, CBG, percentage or potency and the weight of the medicinal cannabis substance in the packaging (All the Data Stored To Memory Of The Centralized Network 466 and Smart Container App 471). Once the law compliant label 490 is scanned, the peripheral smart portable computing device 462 may “unlock” and download the exclusive light color (“The Sky-Blue color assigned to the appetite increasing strain”) and patterns assigned to the “NEW” strain when they have the Smart Container Custom software/application 471 and are connected to the network 466 and via the communication link 486. Once the colors are “unlocked” and downloaded, the peripheral smart portable computing device 462 may manipulate the smart medicinal substance container LED lights 460 and allow the child resistant medicinal substance container 300 to emit the “VIP color” and “color flashing patterns”. Again, this “VIP light” color (“Sky Blue”) and patterns would remain exclusive to those who unlock and download them after scanning a barcode on a label 490 created by the centralized network system 466 and that have the software application 471 installed on their Peripheral smart portable computing device 462. The Network created labels 490 may have two sets of numbers the “Batch” Barcode I.D. number and/or a “single use” Barcode I.D. Number. The smart container's centralized tracking monitoring network 466 may be programed by those designing and managing the smart container centralized computer Network 466, and smart container customized Software Application 471 to only allow the child resistant medicinal substance container 300 authorized, and authenticated to connect to the centralized network 466 via a communication link 484 to emit the exclusive “VIP Light”, “Authenticating Light Color”. The “VIP color” is only accessible when a person purchases a licensed grower's authentic product in the authentic cannabis packaging that have an authentic Centralized Network Law Compliant Label 490 which contains the code for the “VIP Color”. The authentic cannabis packaging may be the first sterile medicinal substance cartridge 320 or the second sterile medicinal substance cartridge 321. The authorized color's data may be programed in a digital or electronic barcoded label 490. When digital or electronic barcoded label 490 can be scanned and recognized by a scanning laser feature or the HD Camera 461 built in the child resistant medicinal substance container 300. Once scanned the child resistant medicinal substance container 300 will be able to access “VIP” color and the LED light/lights 460 to shine and emit the “VIP” color. Only those persons with genuine or authorized first sterile medicinal substance cartridge 320 or second sterile medicinal substance cartridge 321 with the digital or electronic barcoded label 490, and smart container customized Software Application 471 may be able to make the Smart container LED light/lights 460 emit the authorized “VIP color”. These digital or electronic barcoded label 490 may be exclusive to VIP customers, members, and be applied to preauthorized sterile medicinal substance container cartridges, capsules, bags or otherwise cannabis substance packaging for licensed sale. (Note) LED Lights 460 may be on the inside components of the child resistant medicinal substance container 300 as well. For example, lights can be embedded in head-holes or ports of the cartridge head 398. These embedded head-holes LED lights (Not Shown) may aim down or in a way configured to light up the container chambers or tubes from above the tubes and inside the cartridge head 398.

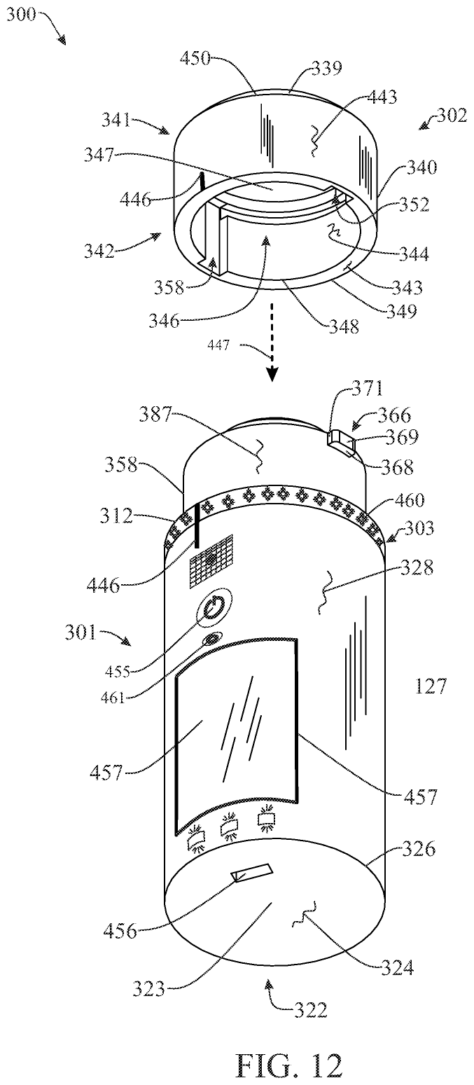

Referring to FIGS. 11-12 & 14, The medicinal substance container domed lid 302 includes a container lid wall 340, a container lid wall top 341, a container lid wall bottom 342, a container lid wall bottom surface 343 a container wall bottom surface inner edge 348, a container wall bottom surface outer edge 349, a container lid wall inner surface 344, a container lid wall outer surface 345, a container lid roof 338, a container lid roof surface 339, a container lid roof surface edge 350, a container lid cavity 346, a container lid cavity ceiling 347, a two dimensional nib sliding channel 350, first vertical nib channel portion 352, a second vertical nib channel portion 356, a circular nib channel portion 160, first vertical nib channel surface A 353, first vertical nib channel surface B 354, first vertical nib channel surface C 355, second vertical nib channel surface A 357, second vertical nib channel surface B 358, second vertical nib channel surface 359 C, a circular nib channel surface 361 A, a circular nib channel surface B 362, a circular nib channel surface C 363, a first nib channel entrance, 364, a second nib channel entrance 365. As best shown in FIGS. 11-12 and 14, the lid roof 338 is dome shaped and is supported by the lid wall 340 at the container lid wall top 341. The container lid roof 338 covers the lid cavity 346 at the container lid wall top 341. The circumference of the lid wall outer surface 145 is equal to circumference of the lid roof surface edge 378 so at the container lid wall top 341, the container lid wall outer surface 345 ends at the lid roof surface edge 378 of the container lid roof 338. The container lid wall outer surface 345 is also equal to the circumference of the cylindrical container body wall outer surface 328 and the circumference of the divider ring wall outer surface 311 so the medicinal substance container 301 has a smooth outer surface. The container lid wall outer surface 345 ends at the container lid wall bottom surface outer edge 349 at the container lid wall bottom 342. The thickness of the container lid wall 340 is equal to the distance between the container lid wall bottom surface inner edge 348 and the container lid wall bottom surface outer edge 349. The container lid cavity 346 of the domed container lid 302 houses the two-dimensional nib sliding channel 350 of the two-dimensional nib sliding channel system 351. The two-dimensional nib sliding channel 350 includes a first and second vertical nib channel portion 352 & 356 and a circular nib channel portion 360. The first and second vertical nib channel portions 352 and 356 are carved out of the container lid wall bottom surface 343 and the container lid wall inner surface 344. The first and second vertical nib channel portions 352 and 356 extending vertically from the container lid wall bottom 342 up toward the container lid cavity ceiling 347. The first left vertical nib channel portions 352 and the second right vertical nib channel portion 356 provide the first nib channel entrance A 364 and the second nib channel entrance B 365 located at the container lid wall bottom surface 343. The plane of the first vertical nib channel surface A 353 and the plane of the first vertical nib channel surface B 354 are parallel to each other and perpendicular to the plane of first vertical nib channel surface C 355 of first left vertical nib channel Portion 352. Likewise, the plane of the second vertical nib channel surface A 357 and the second vertical nib channel surface B 358 are parallel to each other and perpendicular to the plane of second vertical nib channel surface C 359 of second right vertical nib channel Portion 356. The two vertical nib channel portions 352 and 356 are equal in size and shape and are opposite each other within the container lid cavity 346. The first and second vertical nib channel portions 352 and 356 stretch vertically until the six vertical nib channel surfaces 353, 354, 355, 357, 358, and 359 end where they meet a plane defined by the circular nib channel surface B 362. The first and second vertical nib channel portions 352 and 356 are perpendicular to the plane defined by the circular nib channel surface B 362. The circular nib channel portion 360 which is carved into the container lid inner wall surface 344 is located just below the ceiling of the lid cavity 347 at the container lid wall top 341.

The first and second vertical nib channels portions 352 and 356 are equal in size and shape and are opposite each other. The distance between the first and second vertical nib channel portions 352 and 356 is equal to the diameter of the container lid wall bottom surface inner edge 348. The container lid wall bottom surface 343 provides the first and second nib channel slide entrance 364 and 365 at the container lid wall bottom 342. The plane of the first vertical nib channel surface C 355 meets the planes of the first and second vertical nib channel surface A 353 and B 354 at a 90-degree angle creating the first vertical nib channel 352. The plane of the second vertical nib channel surface C 355 meets the planes of the second vertical nib channel surface A 357 and B 358, at a 90-degree angle creating the second vertical nib channel portion 256. The second vertical nib channel surfaces A 357 and B 358 are opposite and a parallel each other. All the surfaces of the two-dimensional nib sliding channel system 351 may be magnetized or formed with circular, ring shaped or bar shaped magnets. All surfaces of the two-dimensional nib sliding channel system 351 may be configured with an armature plate (Not Shown) designed and orientated to be attracted to an electromagnet to strongly resist the container lid 303 from being rotated or removed. Note: The smart medicinal substance container lid 301 may be fashioned to be totally transparent with glass or plastic or be partially transparent. The Lid 301 may be configured in different colors with any of the colors mentioned above or herein. (NOTE) There is another variation of the lid 301 (Not Shown). This lid 527 (Not Shown) may be configured with multiple chambers within it. This lid 527 (Not Shown) may have different sized cavities like the container lid cavity 146 of the container lid 301. These cavities may be configured to be closed/sealed. This lid 527 is similar to the container lid 302 but may be configured with sectioned chambers. The lid 527, like container lid 301, may be manufactured with different colors that can be coordinated to indicate different purposes. For example, the colors may be used to identify a particular medicinal substance's classification in the container. The Lid 527 may double as a cannabis flower display cup, container, capsule, or bowl. The Lid 527 (Not Shown) may be displayed in a lockable product display case traditionally used by jewelry retailers and medical cannabis dispensaries. The Lid 527 (Not Shown) may be configured with a two-dimensional nib sliding channel like the container lid 301 two-dimensional nib sliding channel 350. The lid 527 may be configured similar to the container lid 302 but come in two separate parts or halves. The lid 527 may have a first top part half 528 and a second bottom part half 529 (Not Shown). The two halves may be configured to screw into or snap onto each other. The container lid 527 second bottom half part 529 may have the similar configuration and all the element featured by the domed medicinal substance container lid 102 so they may receive the two nib keys 366 and 372 and fit on and lock to the smart medicinal substance container body 301 and or the divider ring 303 to seal the child resistant medicinal substance container 300 like container lid 302. The roof of the first bottom part half # may be flat vs domed and doubles as a display stage or pedestal to rest cannabis flower # on it to be displayed for sale in a smart electronic display case. The flat surface pedestal or roof of the first bottom part half 528 may be configured with screwing threads # or snapping grooves (Not Shown) at the edges # of the flat roof designed to receive or couple with the second top part half 529 of the lid 527. The second top part half of the 529, may be domed shaped and made with a see-through plastic or glass. The second top part half 529 may be like a bottomless domed shaped half capsule shape (Like the top half of the plastic Easter eggs that when opened, have candy or a prize inside). The capsule # or second top part half 529 is designed to cover a sample of cannabis flower or other cannabis substance placed on the roof, pedestal, or display stage of the first bottom part half 528. The dome shaped capsule or second part half 529 may be configured with a threaded or snapping element configured on or to its circular wall's surface that is designed to screw it into or snap it into the roof of the first bottom half part 528 and cover or encapsulate the sample area substance where cannabis may be displayed for sale. In other words, the glass dome is designed to encapsulate whatever medicinal substance placed on the pedestal or display stage like a water filled snow dome, but the glass dome can be snapped or screwed on or off. The second top half part may be made with thin recyclable plastic, cannabis plastic or even made disposable. They may come configured complete with their own floor or surface that acts as a pedestal or display stage for a cannabis product or flower. The second top half part half 529 may be manufactured as individuals specifically for distribution and sale of cannabis products. They may be used as a waterproof, crush proof hermetically sealed plastic container packaging for a cannabis flower or cannabis product. They may be made crush proof by any method known in the art to make a plastic container crush proof. The second top half part 529 or displaying capsule may be used to sell pre weighed cannabis flower or other cannabis products. These other products include but are not limited to cannabis infused wax, butter, oil, or candies sold by the gram or other measuring unit. These capsules 529 may have labels with the specific labels required by law to be placed on cannabis product packaging. They may look and be packaged like the plastic Keurig coffee and tea disposable capsules used in the electronic Keurig brewing machines. The capsules may contain one, two, three point five, or seven grams of a cannabis flower or cannabis product and be ready for lawful licensed cannabis distribution and sale. This design is so clear container lid glass or plastic domed capsules may be displayed securely on the child resistant medicinal substance container 300. A customer or patient may examine the samples and know/identify the cannabis flower locked inside the first sterile refillable medicinal substance cartridge 320 cavity or the second sterile refillable medicinal substance chamber 321 of the child resistant medicinal substance container 300 without the need to open it. This lid 527 acts as a quick access to cannabis flower also without need to use the passcode to open the container. These capsules or top halves may have perforated plastic domes or glass domes so the cannabis or other substance inside may be sniffed, and the smell of the cannabis flower can be sensed/experienced.

The first sterile refillable medicinal substance cartridge 320 of the medicinal substance cartridge assembly 319 is cylindrically shaped. The first refillable medicinal substance cartridge 320 is sized and shaped to be inserted and slid into the container body cavity 335 to be secured and retained thereby with a seatbelt like click locking sound. The first sterile medicinal substance cartridge may be popped, launched, or ejected out of the medicinal substance container body cavity via a spring like launching system composing of multiple strong tiny springs (Not Shown) configured with circulator container body base inner surface cavity floor 325. The launching system may be electronically activated via user interface and microchip connected sensors, or mechanically activated via a spring-loaded catapulting system with a physical button or switch that releases the springs tension. The ejection of the medicinal substance cartridges 320 and 321 may also be realized via a magnetic force controlling system whereby the circulator container body base inner surface cavity floor 325 has two magnets or a magnetic surface is configured on, behind, or within the circulator container body base inner surface cavity floor 325 and another two magnets or a magnetic surface is configured on, behind, or within the first refillable medicinal substance cartridge base outer surface 382. The respective magnets and or magnetic surfaces may be configured to repel each other's magnetic force thereby pushing, launching, or ejecting the first sterile refillable medicinal substance cartridge 320 from the cylindrical container body cavity 335. It is to be noted that the force and or the magnetic polarity of the magnets used may be electronically reversed thereby causing the first refillable medicinal substance cartridge 320 to be pulled towards and attracted to the circulator container body base inner surface cavity floor 325 of the circular container body base 322. (Note) The smart medicinal substance container's 300 magnetic field cartridge ejecting system may be powered by any method known in the art to use magnets to pull or push a device in and out of a cavity, receptacle or flatform. The first sterile refillable medicinal substance cartridge base edge 384 is lined up with the divider ring wall top inner edge 313. The first sterile refillable medicinal substance cartridge 320 is then pushed to slide into the container body cavity 335 until it contacts the container body base inner surface cavity floor 325. The first refillable medicinal substance cartridge 320 may be secured to or attached to container body base 332. They can be secured by glue, tape, or another adhesive. They may also be detachably secured by way of fasteners such as screws pressed pins, or any other fasteners known in the art if they allow the two elements to be attached and secured to each other. The two elements may also be designed with a locking mechanism (Not Shown) allowing the two elements to lock to each other. The locking mechanism may be a key and groove system similar to the two-dimensional slide channel system 351 describe above in which a twisting and lifting movement is used to detach and remove the first refillable medicinal substance cartridge 320 from the cylindrical medicinal substance container body 301 when a channel or groove in the container body 301 (Not Shown) is configured to receive a nib key configured to the base 322 of first sterile refillable medicinal substance cartridge 320 allowing the nib key to slide on and in the channel or groove designed in the container body 301 (Not Shown). (NOTE) The first sterile refillable medicinal substance cartridge 320 may be sized and shape so when it is inserted into the smart container body cavity 335, the first medicinal substance cartridge wall outer surface 387 slides against the cylindrical container body wall inner surface 329 with some friction but providing a small crevice. When the first sterile refillable medicinal substance cartridge 320 is injected and slid into the container body cavity 335, the air that's in the container body cavity 335 is forced out from the small crevice that's created between the first medicinal substance cartridge wall outer surface 387 and the cylindrical container body wall inner surface 329. when the first sterile refillable medicinal substance cartridge 320 enters and begins to fill the cavity 335. Once the first sterile refillable medicinal substance cartridge 320 is inserted and fully occupies the container body cavity 335 where the expelled air once occupied, a suction effect may be created as air is pulled back through the crevice and begins build pressure inside the container body cavity 335 and fill the void the first sterile refillable medicinal substance cartridge 320 is creating as it vacates the container body cavity 335. When the first sterile refillable medicinal substance cartridge 320 is fully removed from the container body cavity 335 the high-pressured air rapidly escaping may cause a popping sound may be heard. The popping sound is like the sound heard when a champagne cork is removed or popped off the champagne bottle. The circumference of the cavity 335 of the smart container body 301 may be slightly greater than the circumference of the first medicinal substance cartridge wall outer surface 387 so when the first medicinal substance cartridge base edge 384 is lined up with the divider ring wall top inner edge 313. The first sterile refillable medicinal substance cartridge 320 may be pushed and slid into the container body cavity 335 until it contacts the container body base inner surface cavity floor 325. The room between the container body 301 and the first refillable medicinal substance cartridge 320 may have a diameter of 32.27 mm and has a first refillable medicinal substance cartridge upper end 379 and lower end 380, a first refillable medicinal substance cartridge base 381, a first refillable medicinal substance cartridge base outer surface 382, a first refillable medicinal substance cartridge base inner surface floor 383, a first refillable medicinal substance cartridge base edge 384, a first refillable medicinal substance cartridge wall 385, a first refillable medicinal substance cartridge wall inner surface 386, a first refillable medicinal substance cartridge wall outer surface 387, a first refillable medicinal substance cartridge wall top 388, a first refillable medicinal substance cartridge wall top outer edge 389, a first refillable medicinal substance cartridge wall top inner edge 390, a first refillable medicinal substance cartridge cavity 391, a first refillable medicinal substance cartridge cavity entrance 392, At the first refillable medicinal substance cartridge upper end 379, a first left side nib key 366 and a second right side nib key 372 extend from the first refillable medicinal substance cartridge wall top outer edge 389 and a first refillable medicinal substance cartridge wall top surface 393. The first left side nib key 366 is located on the left side of the first refillable medicinal substance cartridge 320 and the second right nib key 372 is located on the right side of the first refillable substance container 320. The nib keys 366 and 372 are opposite each other and designed to align with the first and second vertical nib channel portion 352 and 356 of the two-dimensional nib sliding channel 150 and be inserted into the first nib channel slide entrance a 364 and second nib channel slide entrances 165. The nib keys 366 and 372 are designed and shaped to slide into and onto the two-dimensional nib sliding channel 350 located under the container lid 302 in the container lid cavity 346. The medicinal substance cartridge assembly 319 may be designed with food grade material and manufactured with sterile methods or designed to be manufactured in law compliant sterilized environments. The sterilization of the medicinal substance cartridge assembly 319 may meet all the requirements regulated by governments and agencies that are authorized to create and or enforce laws governing medical devices or substances intended for human consumption. The medicinal substance cartridge assembly 319 may be labeled with any cannabis regulation compliant label disclosed herein. The first refillable medicinal substance cartridge 320 may be designed with stainless steel or be designed to have stainless steel surfaces. (NOTE) The medicinal substance cartridge assembly 319 and the child resistant medicinal substance container 300 may be hermetically sealed and have gasket (NOT SHOWN) place between the lid 302 and the container body 301 to help prevent the odor of the substance in the child resistant medicinal substance container 300 from escaping when the lid 302 is on the child resistant medicinal substance container 300. The Gasket may be made from a pliant material including but not limited to silicon, rubber, foam, or any other pliant material used to make gaskets to seal lids, doors, windows, medical device etc.

The second sterile refillable medicinal substance cartridge 321 may be 99.06 mm tall and sized, shaped, and designed to be inserted into the first refillable medicinal substance cartridge 320 and be retained thereby. The second refillable medicinal substance cartridge 321 includes a second refillable medicinal substance cartridge head 398, a first cylindrical substance chamber 394, a second cylindrical substance chamber 395, a third cylindrical substance chamber 396, a fourth cylindrical substance chamber 397 configured to and extending from the refillable medicinal substance cartridge head 398. The cylindrical substance chambers may be equal in shape and size. The second refillable medicinal substance cartridge head 398 may have a diameter of 32.27 mm and may be configured with sensors, a microcontroller or processor, LED Light/Lights, a camera in or on it (Not Shown). The second refillable medicinal substance cartridge head 398 has a second refillable medicinal substance cartridge head domed top surface 340, a second refillable medicinal substance cartridge head bottom flat surface 341, a second refillable medicinal substance cartridge head curved side surface 443, a second refillable medicinal substance cartridge head center point 342, a second refillable medicinal substance cartridge head first head hole 306, a second refillable medicinal substance cartridge head second head hole 315, a second refillable medicinal substance cartridge head third head hole 235, a second refillable medicinal substance cartridge head fourth head hole 335. The four head holes 306, 315, 325, and 335 respectively each have a head hole inner surface, a first head hole inner surface 307, a second head hole inner surface 316, a third head hole inner surface 326 and a fourth head hole inner surface 333. The four head holes 306, 315, 325, and 335 are the respective entrances to each of the four cylindrical substance chambers 394, 395, 396, and 397. The four head holes 306, 315, 325, and 335 may be numbered or have another identifying markers on the surface of the medicinal substance cartridge head 398 for each of the head holes (Not Shown). The head holes 306, 315, 325, and 335 bore straight through the second refillable medicinal cartridge head 321. Medicinal substances placed in the second sterile refillable medicinal substance cartridge 121 may enter through the cartridge head holes 306, 315, 325, and 335. The head holes may be sized and shaped equal to the cylindrical substance chambers 394, 395, 396, and 397. The four head holes 306, 315, 325, and 335 and their respective entrances to each of the four cylindrical substance chambers 394, 395, 396, and 397 may be configured with sensors, cameras, retractable cavity doors. The four head holes 306, 315, 325, and 335 are the respective entrances to each of the four cylindrical substance chambers 394, 395, 396, and 397 may be sealed with any Child-Resistant Seal known in the art. The second refillable medicinal substance cartridge head 398 may be configured with head hole sliding doors (Not Shown). The four head holes 306, 315, 325, and 335 may each have a head hole sliding door that may be used to slide over their respective head hole to close the four head holes 306, 315, 325, and 335. The sliding doors may use a detent mechanism with bumps and recesses to where an element is pressed or pulled in order to enable the sliding doors to be unlocked and slid open or closed. Shafts may be formed in the medicinal substance cartridge head where the sliding doors slide in when opened, and out of when the head hole doors are closed. There may be more than four head holes and chambers. For example, there may be 6, 8, 10 or more holes in the second refillable medicinal substance cartridge head 398. The holes may be aligned symmetrically and equally around a center point of the second refillable medicinal substance cartridge head 398. The four head holes 306, 315, 325, and 335 may have a diameter of 10.44 mm or Smaller.

The first cylindrical substance chamber 306 has a first chamber base 399, a first chamber base edge 302, a first chamber wall 303, a first chamber wall outer surface 205, a first chamber wall inner surface 204, a first chamber base inner surface floor 301, a first chamber opening (Not Shown). The first chamber base 399 may be configured to be removed or detached from the first cylindrical substance chamber 306 for cleaning. The removable may be realized by unscrewing the first chamber base 399 from the substance chamber 396. The first chamber base 199 and the cylindrical substance chamber 306 may be outfitted with threads (Not Shown) so they can screw to each other. The ability for the first chamber base 399 to separate from the cylindrical substance chamber 306 is crucial to clean the first chamber base 399. The second cylindrical substance chamber 196 has a second chamber base 308, a second chamber base edge 311, a second chamber wall 312, a second chamber wall outer surface 314, a second chamber wall inner surface 313, a second chamber base inner surface floor 310, a second chamber opening (Not Shown). The second chamber base 396 may be configured to be removed or detached from the first cylindrical substance chamber 308 for cleaning. The removable may be realized by unscrewing the first chamber base 308 from the substance chamber 396. The second chamber base 308 and the cylindrical substance chamber 396 may be outfitted with threads (Not Shown) so they can screw to each other. The ability for the second chamber base 399 to separate from the second cylindrical substance chamber 396 is crucial for clean the first chamber base 399. The third cylindrical substance chamber 396 has a third chamber base 318, a third chamber base edge 321, a third chamber wall 322, a third chamber wall outer surface 324, a third chamber wall inner surface 323, a third chamber base inner surface floor 320, a third chamber opening (Not Shown). The third chamber base 318 may be configured to be removed or detached from the third cylindrical substance chamber 396 for cleaning. The removable may be realized by unscrewing the third chamber base 308 from the third cylindrical substance chamber 396. The third chamber base 318 and the third cylindrical substance chamber 396 may be outfitted with threads (Not Shown) so they can screw to each other. The ability for the third chamber base 318 to separate from the third cylindrical substance chamber 318 is crucial for clean the first chamber base 399. The fourth cylindrical substance chamber 397 has a fourth chamber base 328, a fourth chamber base edge 331, a fourth chamber wall 332, a fourth chamber wall 332, a fourth chamber wall inner surface 333, a fourth chamber base inner surface floor 330, a fourth chamber opening (Not Shown). The fourth chamber base 397 may be configured to be removed or detached from the fourth cylindrical substance chamber 397 for cleaning. The removable may be realized by unscrewing the fourth chamber base 397 from the fourth cylindrical substance chamber 397. The fourth chamber base 328 and the fourth cylindrical substance chamber 397 may be outfitted with threads (Not Shown) so they can screw to each other. The ability for the fourth chamber base 328 to separate from the fourth cylindrical substance chamber 397 is crucial for cleaning the fourth chamber base 328.

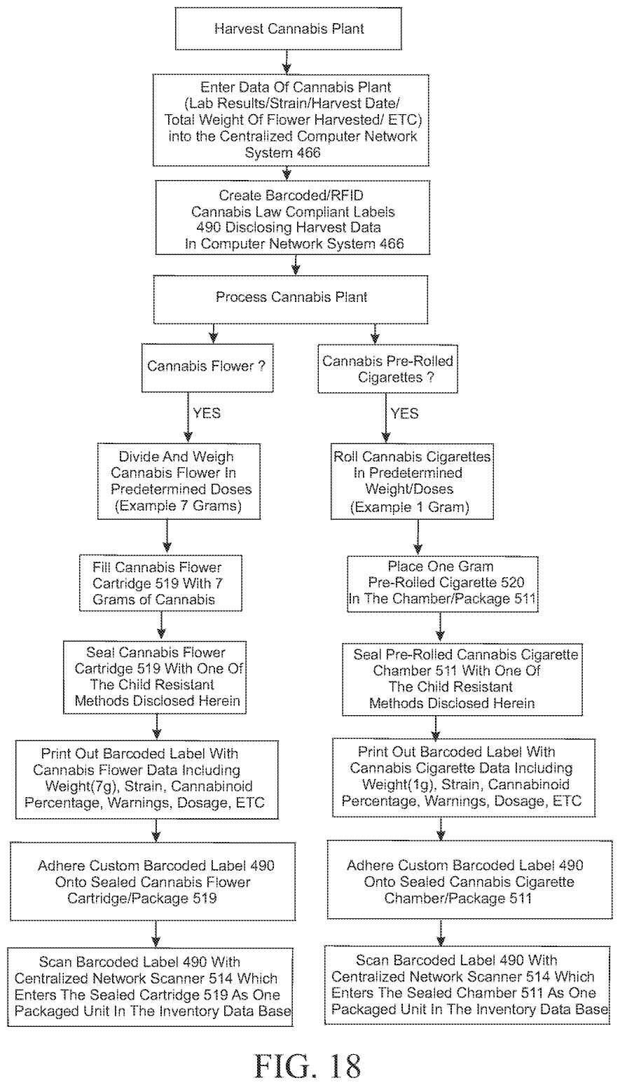

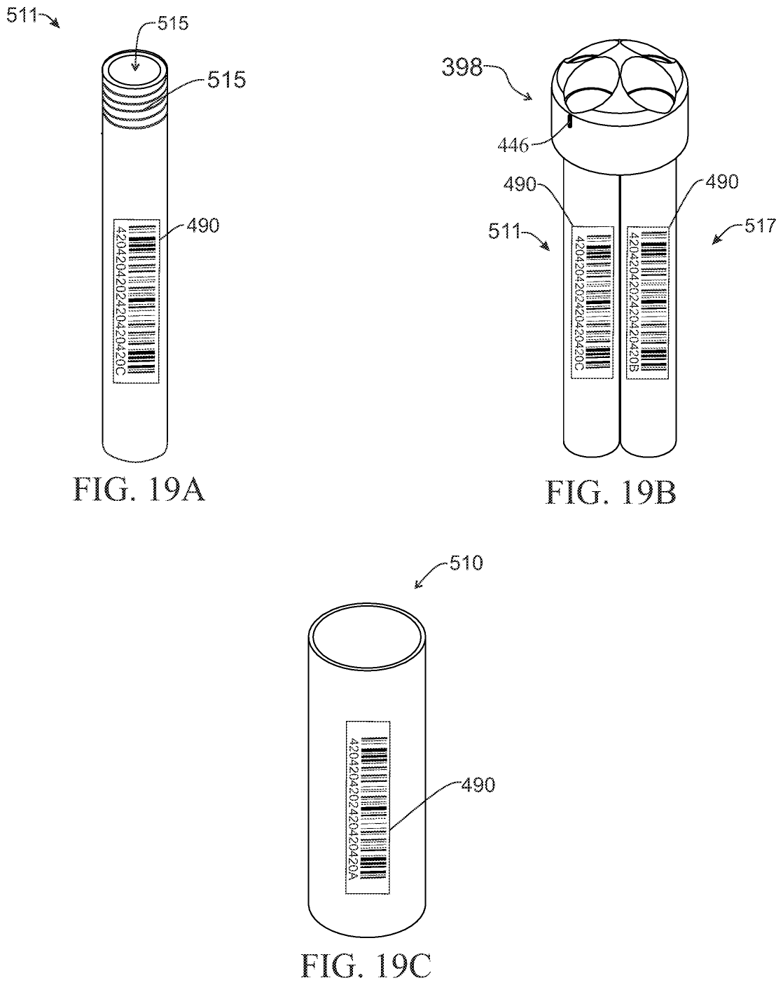

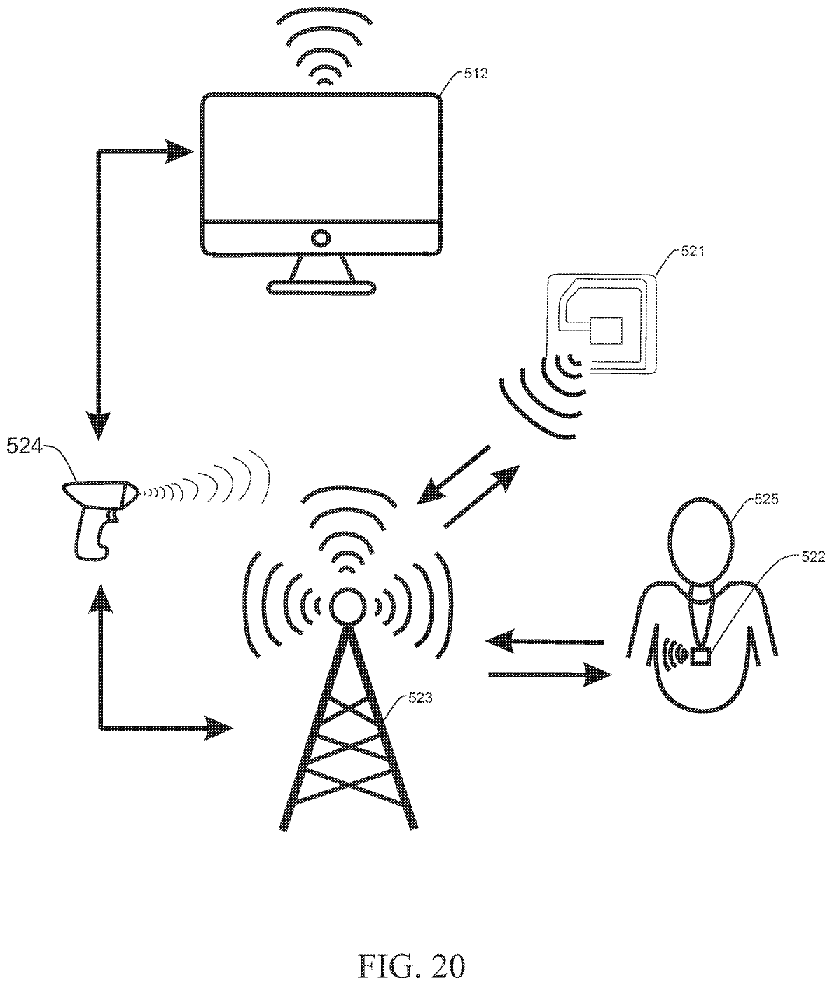

The first medicinal substance cartridge head hole 306, the second the cartridge head hole 315, the third cartridge head hole 324, and the fourth cartridge head holes 335 lead to the four cartridge chamber entrances (Not Shown) located below the cartridge head 398. The second refillable medicinal substance cartridge head 398 is configured to attach at the second refillable medicinal substance cartridge head bottom flat surface 341 to the four cylindrical substance chambers 394, 395, 396, and 397. The second refillable medicinal substance cartridge head 398 is configured to be attached in a manner where the four chamber openings (Not Shown) are aligned with the four cartridge head holes 306, 315, 325, and 335. The medicinal substances placed in the second refillable medicinal substance cartridge 321 enter through the cartridge head holes 306, 315, 325, and 335. The head holes are sized and shaped equal to the four cylindrical substance chambers 394, 395, 396, and 397. The second refillable medicinal substance cartridge head 398 and the four cylindrical substance chambers/tubes 394, 395, 396, and 397 may be formulated as one unit. The four cylindrical substance chambers/tubes 394, 395, 396, and 397 may be configured to attach to bottom of the cartridge head 398 via screwing a thread 515 designed to screw into threaded fastener holes (Not Shown) bored into the cartridge head bottom surface 341. Once the cylindrical substance chambers 394, 395, 396, 397 are screwed or secured to the second refillable medicinal substance cartridge head 398. The four base surfaces 300, 392, 319, and 328 of the cylindrical substance chambers 394, 395, 396, and 397 are placed at and within the circumference of the first medicinal substance cartridge cavity top inner edge 390. The second refillable medicinal substance cartridge 321 is then pushed and slid into the first refillable medicinal cartridge cavity 391 to be retained thereby. Once the second refillable medicinal substance cartridge 321 is fully inserted into first refillable medicinal substance cartridge 320, the medicinal substance container lid 302 may be placed on. (Note: The Second refillable substance cartridge 321 may be removed and the user may elect to use the container with just the first refillable medicinal substance cartridge 320. The cavity 391 of the first refillable medicinal substance container 320 has more volume and can contain other shaped capsules, containers, or cartridges (like small quarter sized wax/butter/oil jars), cannabis flowers, cannabis oils, cannabis butters, cannabis infused candies, or any other cannabis product. The first sterile refillable medicinal cartridge 320 also can hold a larger capacity of cannabis cigarettes or cannabis cigars.) The first refillable medicinal substance container 320 may also be configured to hold a closed container #(Not Shown) filled with cannabis oil, butter, or wax whereby the second refillable substance cartridge may rest upon the container when fully inserted into the first refillable medicinal substance cartridge 320. The container may be like those used in lip balm packaging where the user dips their finger in plastic or small glass jar or container to apply the lip balm to their finger and then lips. The cannabis wax or cannabis butter jars or small containers having lids that screw on and off and are sized and shaped to be received by the medicinal substance container body 301 or the first sterile refillable medicinal substance cartridge 320. (NOTE) A custom child resistant cartridge style packaging for cannabis infused Candy 510 (See FIGS. 18 & 19 may be designed sized and shaped to retain cannabis infused “edibles”/candies such as lemon drops, nerds, gummy bears, hard candies, and the like. The cannabis infused candy packaging/cartridge 510 may be designed to be inserted in the child resistant medicinal substance container 300 in a manner like the first refillable medicinal substance cartridge 320. The cannabis infused candy packaging/cartridge 510 may be sealed with any child resistant method meeting and complying with the Poison Prevention Packaging Act (PPPA) included but not limited to “Single Use” and Multiple Use”. (NOTE) The four cylindrical substance chambers/tubes 394, 395, 396, and 397 of the second refillable medicinal substance cartridge 321 may be “tagged” individually using the centralized network system 466. A first cylindrical substance chamber/tube/package 511 (See FIGS. 18 & 19A) may be used to “package” a single pre-rolled cannabis cigarette 512 (Not Shown). The first cylindrical substance chamber 511 may have a first cylindrical substance chamber/packaging opening or entrance 515 (See FIGS. 18 & 19A). The first cylindrical substance chamber entrance 515 may be sealed with any child resistant method (NOT SHOWN) meeting and complying with the Poison Prevention Packaging Act (PPPA) included but not limited to “Single Use” and Multiple Use”. The first single cylindrical substance chamber/package 511 may be “labeled” with a cannabis law compliant Barcoded label 490 created by the centralized network system 466 placing the law compliant labeled packaged and its contents into “inventory”. The first single cylindrical substance chamber/package 511 may be designed with a thread 516 on the top or bottom to screw into the bottom of the second refillable medicinal substance cartridge head 398. The single cylindrical substance chamber/package 511 would screw into the second refillable medicinal substance cartridge head bottom flat surface 341 by way of threads (NOT SHOWN) bored into the second refillable medicinal substance cartridge head bottom 439. FIGS. 18 & 19B shows the first cylindrical substance chamber/package 511 and a second single cylindrical substance chamber/package 517 fully screwed into the second refillable medicinal substance cartridge head 398 with the law compliant label 490 applied to them. (NOTE) An element 513 (NOT SHOWN) may be designed in the cartridge head 398 that punctures the seal once the single cylindrical substance chamber/package is fully screwed into the second refillable medicinal substance cartridge head 398. Once a cylindrical substance chamber is fully screwed in, the cannabis cigarette 512 may be accessible via one of the four cartridge head holes/ports 306, 315, 325, and 335. Although, the drawings show the second refillable medicinal substance cartridge head 398 designed with four head-holes or ports, it is understood that they may be designed to have 1, 2, 3, 4, 5, 6, 7, 8, 9, 10, or more head holes to accommodate multiple pre-rolled cigarettes 512.

As best shown in FIGS. 3A & 3B, once the second medicinal substance cartridge 321 and the first medicinal substance cartridge 320 are fully engaged and inserted in the medicinal substance container body 301, the container lid 302 may be secured to the body. a first left side nib key front surface 369 contacts the first vertical nib channel surface C 355, a first left nib key right side surface 371 contacts the first vertical nib channel surface B 354 and a first nib key left side surface 370 contacts the second vertical nib channel surface A 353. Once inserted, a second nib key front surface 375 contacts the second vertical nib channel surface C 359, a second nib key right side surface 377 contacts the second vertical nib channel surface B 358 and a first nib key left side surface 376 contacts the second vertical nib channel surface A 357. The respective surfaces of the nib keys 366 and 372 contact the respective surfaces of the first and second vertical nib channel portion 352 and 356 in a way that enables the first and second nib keys 366 and 372 to slide on an up the first and second vertical nib channel portion 352 and 356. The diameter of the first refillable medicinal container cartridge top outer edge 389 is slightly smaller than the diameter of the container lid wall bottom surface inner edge 348 enabling the first refillable medicinal container cartridge wall top 388 and a portion of the first refillable medicinal substance cartridge wall outer surface 387 to slide up into the container lid cavity 346 When the Nib keys 366 and 372 are aligned with the vertical nib channel portions 352 and 356. The top of the first refillable medicinal substance cartridge wall outer surface 387 then engages with and slides up against the container lid inner wall inner surface 344. The first and second nib keys 366 and 372 slide up until the first and second nib key top surfaces 367 and 373 cross the plane defined by the circular nib channel surface B 362 of the circular nib channel portion 360 and contacts the circular nib channel surface A 361 where nib keys 166 and 372 are stopped from sliding vertically any further. Once the nib keys top surfaces 367 and 373 contact the circular nib channel surface A 361, and the container lid wall bottom surface 343 contacts the divider ring wall top surface 106, The user can turn the container lid 302 clockwise or counterclockwise engaging the first and second nib key bottom surfaces 367 and 374 with the circular nib channel surface B 362. The circular nib channel portion 360 designed to slide on and around the nib keys 366 and 372. Once the container lid is rotated enough to where the nib keys 366 and 372 are unaligned with the first and second vertical nib channel portions 352 and 356, the container lid 302 is secured or locked onto the container body 301. A user can than turn the container lid 302 rotating the circular nib channel portion 360 around the first and second nib keys 366 and 372 around the circular nib channel 360 until the first and second nib keys are aligned once again with the vertical nib channel portions 352 and 356. Once aligned, the user can pull the container lid 302 upward and removed it thereby opening the child resistant medicinal substance container 300. Note: alternatively, the nib keys 366 and 372 may be built on a structural element coupled to the cylindrical container body 301. For example, the nib keys 366 and 372 may be attached to a first and a second raised nib key supporting structures opposite each other and connected to the container body wall inner surface 329 of the cylindrical container body 301. The first and a second raised nib key supporting structures raising above and pass the cylindrical container body wall top surface 331 and positioned inside the cylindrical container body wall top surface inner edge 333. With this configuration the child resistant medicinal substance container 300 can be locked with or without the first medicinal substance cartridge 320.

There is now disclosed an electromagnetic locking system 600 including but not limited to an electromagnet and armature plate lock 489 having an electromagnet 602 and an armature plate 603, a set of current or voltage wires 606. The current or voltage wires 606 connected to the electromagnet and armature plate lock 489. The magnetic field or pull or push force of the electromagnets 602 can be amplified, decreased, or reversed with the touchscreen interface of a peripheral smart mobile computing device such as a smart phone, tablet, or laptop computer paired with the child resistant medicinal substance container that has downloaded and installed the smart child resistant medicinal substance container custom software application 471 designed for the smart child resistant medicinal substance container 300 and its systems 499. The force of the magnetic field of the electromagnet and armature plate lock 489 can be increased or decreased via the voltage sent to the electromagnet and armature plate lock 489. The electromagnet and armature plate lock 489 may be installed on or within any surface of the two-dimensional nib sliding system 351. Including but not limited to the first and second vertical nib channel portions 352 & 356 or/and the circular nib channel portion 360. For example, a portion or the whole surface of the circular nib channel sliding portion 360 may be the electromagnet 602 and the first and second nib keys 366 and 372 may function as the armature plate 603. The electromagnet 602 and the armature plate 603 designed and orientated to be attracted to each other strongly enough to keep the container lid from being rotated or removed when the electromagnetic locking system 600 is armed and the child resistant medicinal substance container 300 is locked. Alternatively, the first and second nib keys 366 and 372 may be the electromagnet 602 and a portion or the whole surface of the circular nib channel sliding portion 360 may be the armature plate 603. All the surfaces of the two-dimensional nib sliding channel system 351 disclosed herein may be made the electromagnet 602 or be configured to function as the armature plate 603 of the electromagnetic locking system 600. The electromagnetic locking system 600 may configured to be a “Fail Safe” electromagnetic locking system where power is required to lock the electronic medicinal substance container lid to the lockable handheld electronic medicinal substance container or configured to be a “Fail Secure” electromagnetic locking system where power is required to unlock the electronic medicinal substance container lid from the lockable handheld electronic medicinal substance container. The electromagnetic locking system 600 may include a lock that includes a retractable latch, bar, or bolt 604, and a strike plate 605. The retractable latch 604 may be a small or micro metal latch or a (small metal protruding bolt) and the strike plate 605 may be small metal plate with a latch or bolt receiving hole). The latch or strike plate (Not Shown) may be installed on or within any surface of the two-dimensional nib sliding channel system 351. The retractable latch 604 may be located on the first sterile refillable medicinal substance cartridge upper end 379 and be positioned on or in the two nib sliding keys 366 and 372. The strike plate 605 may be in or on any surfaces of the two-dimensional nib sliding channel system 351 defined herein. The strike plate 605 may be micro or small enough to fit within or on a 3 mm channel or surface. By the retractable latch 604 being positioned on one element of the container body, and the strike plate being position on one element of the container lid 302 or vice versa, the retractable latch 604 can be made to be push, pulled, or pressed into the strike plate 605 thereby locking and preventing the container lid from being rotated or alternatively retracting and thereby unlocking or allowing the container lid 302 to be turned and removed. The child resistant medicinal substance container 300 by way of the medicinal substance monitoring system 499 can connect and monitored by and on a centralized encrypted smart container centralized computer network 466 and data cloud 512. The child resistant medicinal substance container 300 is connected to the centralized encrypted smart container centralized computer network 466 and data cloud 512 by way of the internet. The centralized encrypted smart container network may be a LAN (local area network), a PAN (personal area network), a MAN (metropolitan area network) or a WAN (wide area network). The centralized encrypted smart container centralized computer network 466 and data cloud 512 may be accessed by an authorized and synced peripheral smart mobile computing device 462 utilized by a patient or a drug rehabilitation community. For example, a cannabis patient community may use the smart container custom software application 471 to log into an account on a website. The patient can rank, leave reviews, and compare different strains of the cannabis or different kinds of cannabis extracts. The smart container custom software application 471 may include a website with a chat room and or blog. The chat room being marketed to a fan club. The website allowing a user to create a VIP Members/Customers account. The centralized encrypted smart container centralized computer network 466 may be linked to for accessed via other industry medicinal cannabis themed website or a social media app. The smart container custom software application 471 may be created to educate patients or other users about medicinal cannabis or other prescribed medicinal products and substances. The website with the chat room and or the blog is where members may to post comments and discuss medicine side effects, medicinal qualities, and overall experiences and opinions about consuming a sponsored or elected cannabinoid product. The smart container custom software application 471 along with the centralized encrypted smart container centralized computer network 466 and data cloud 512 may also be used to manage, identify, track, store, and record data relevant to any substance placed in the child resistant medicinal substance container 300 by way of the barcode 490 placed on the first and second sterile medicinal substance cartridges 320 and 321. The smart container custom software application 471 along with the centralized encrypted smart container centralized computer network 466 and data cloud 512 may track and monitor one lockable smart electronic handheld portable medicinal cannabis substance container or a paired unified batch of them. The medicinal substance monitoring system 499 of the paired unified batch of containers would be synced and paired with the smart container custom software application 471 along with the centralized encrypted smart container centralized computer network 466 and data cloud 512 with Wi-Fi, Bluetooth, RFID, radio wave or other signal sending and receiving technology known in the art. The integration of one or a unified paired group of child resistant lockable medicinal substance containers 300 using the smart container custom software application 471 along with the centralized encrypted smart container centralized computer network 466 and data cloud 512 enables medical professionals, licensed cannabis cultivators, licensed patients, licensed retailers and or government cannabis regulating agencies to track the location, rate of consumption, accessibility, volume or weight of a prescribed cannabis medical substance or the containers that they are packaged and transported in is novel and useful. The centralized network may monitor the day and the time the smart medicinal container or a smart bag was filled or emptied and who opened it.

The child resistant medicinal substance container 300 may record data showing when and where a person contacts the child resistant medicinal substance container 300. The data can be attained by using medicinal substance monitoring system 499 which includes a light sensor 476, a vibration/motion sensor 477, a sound sensors 478, a GPS modules 46,3 High-Definition Cameras 461, computerized digital scale 479, RFID signal transmitters and signal receivers 521, a processor controller 468, Wi-Fi module 480, Bluetooth module 474, Micro-speaker 472 Wired Interface 503, Microphone 463, Battery, Magnetic Lock, Memory 467 and digital clocks/calendars that work together to record the day, time, and location of when a person may have made contact with the smart child resistant medicinal substance container 300. This data may be stored in the container memory 467 and uploaded to the smart container centralized computer network 466 and data cloud system server 512 for storage. Those growing, processing, and bagging or filling or otherwise handling legally grown licensed cannabis may also outfit their employees 525 with smart wearables device 522. The smart wearable device may be in the form including but not limited to name tags, smart watches, smart rings, smart bracelets configured with RFID software and technology. These smart wearables device 522 may be activated, registered, synced, and paired with and to the smart container centralized computer network 466 and data cloud system server 512. Using the smart container centralized computer network 466 and data cloud system server 512, the smart containers allow a record to be kept of when the smart child resistant medicinal substance container 300 that is linked to the smart container centralized computer network 466 is handled or opened by a person wearing the RFID tag technology 522. The smart container centralized computer network 466 also monitors and records if the child resistant medicinal substance container 300 is moved, handled, opened, locked, Unlocked, or loaded with the sterile medicinal substance cartridge 320 or 321. With the computerized digital scale 479, the sterile medicinal substance cartridges 320 or 321 can be weighed thereby weighing the prescribed medical substance stored in them. The digital scale 479 including pressure sensors to measure the weight of the medicinal substance in the first sterile medicinal substance cartridge 320 or the second sterile medicinal substance cartridge 321 once either of them is inserted and slid into the cylindrical medicinal substance container body 301. The digital scale 479 can be programmed to weigh the refillable medicinal substance cartridges 320 or 321 before they are removed, and again the next time the refillable medicinal substance cartridges 320 or 321 is inserted again. The weight of the first sterile medicinal substance cartridge 320 and the second sterile medicinal substance cartridge 321 are predetermined and recorded into the smart lockable container authenticating, securing, and tracking Medicinal substance monitoring system 499. The child resistant medicinal substance container 300 uses the computerized digital scale 479 to weigh the first sterile medicinal substance cartridge 320 or the second sterile medicinal substance cartridge 321 when they are inserted into the cylindrical medicinal substance container body 301. The computerized digital scale 479 is position on the surface of the circular container body base inner surface cavity floor 525 so when the refillable medicinal substance cartridges 320 or 321 make contact with the computerized digital scale 479 and the pressure sensors are triggered the computerized digital scale 479 weighs the refillable medicinal substance cartridges 320 or 321 recording and sending the weight data to the processor controller 468, memory 467 and the smart container centralized computer network 466 and data cloud system 512. The date and time the weight data is also recorded to the system memory 467 and 512. By this method, the child resistant medicinal substance container 300 can record the weight or number of cannabis seeds, cannabis flower, cannabis cigarettes, cigars, pills, capsules, or liquid contained in the child resistant medicinal substance container 300 in real time. The computerized digital scale 479 has a tare button or feature used to zero out the weight after the refillable medicinal substance cartridges 320 or/and 321 are inserted and contact the computerized digital scale 479. The weight measuring system may also have a feature to change the units of measure. For example, the computerized digital scale 479 can be changed to weigh grams, ounces, drams, liters etc. the smart container centralized computer network 466 and data cloud system 512 records when the weight unit was change and who changed it by receiving signals and images from the RFID Chip 522, High-definition camera 461 or other identifying technology. The smart container centralized computer network 466 and data cloud system 512 is configured to work with the medicinal substance monitoring system 499 to weigh and identify substances and record identifying qualities and facts of a particular cannabis medicinal substance secured in the child resistant medicinal substance container 300 like substance's consistency, chemical makeup, solid or liquid form, if the substance is a powder, if the substance is a pill or capsule, or the size and shape and of a substance and record it to memory 512, 465, and 467. The smart container centralized computer network 466 may be programed to alert persons authorized to log on to the smart container centralized computer network 466 and monitor the child resistant medicinal substance container 300 and be notified of a certain event or events. For example, system 466 may be programed to alert the user or a medical professional when the child resistant medicinal substance container 300 is being tempered with, opened too frequently at unprescribed/unauthorized times, or at an unauthorized location. The memory 467 and the smart container custom software application 471 controlled by the processor 468 allows the container to be programed to lock or unlock itself at the occurrence of a particular event or at certain scheduled times arranged by an authorized user, Licensed Cannabis Cultivator, Licensed Cannabis Patient, Licensed Cannabis Dispensary, Medical professional, Patient or Caregiver. The locking and unlocking of the child resistant medicinal substance container 300 can be programed or configured to be activated by the sense of the light sensor 476, motion/vibration 477, and sound sensor 478 located in or on the child resistant medicinal substance container 300. The sensors can be the light sensor 476, motion/vibration 477, and sound sensor 478, or a humidity sensor, a mildew sensor or any other sensor known in the art. The electronic magnetic locking mechanism 600 may be powered by a magnetic field's push or pull. The magnetic force may be configured to increase or decrease by sending or supplying voltage to the electromagnetic locking system 600. For example, if the smart child resistant medicinal substance container 300 is in a medicine cabinet or secured in a dark humidity and temperature controlled storage room where light is absent or low, if an individual opens the medicine cabinet or enters and turns on a light in the storage room allowing light to enter the cabinet or room and contact or shine on the container's light sensor 476, computerized electromagnetic locking system 600 may be programmed to lock the child resistant medicinal substance containers 300. The light sensor 476 sends a signal to the processor, the processor 468 sends a signal to the electromagnetic locking system to lock the container lid 302. The medicinal substance monitoring system 499 may send an alert data signal to and throughout the smart container centralized computer network 466 and data cloud system 512, to be recorded and translated. The alert data signal may also be sent to be received by the smart mobile computing device 462 or any other computing device connected to the smart container centralized computer network 466 and data cloud system 512 and authorized to receive data from the network. Another example, if the smart child resistant medicinal substance container 300 is placed on the counter and sitting still, the motion/vibration sensor 477 configured on or in the smart child resistant medicinal substance container 300 may detect when an individual touches, moves, or picks up the smart child resistant medicinal substance container 300. The smart child resistant medicinal substance container 300 may identify the person wearing the company prescribed RFID configured wearable identification tag device 522. The smart child resistant medicinal substance container 300 can then lock itself (secure the lid) if the person is not authorized to touch the smart child resistant medicinal substance container 300 and send an alert to and through the smart container centralized computer network 466 and data cloud system 512 that an unauthorized event has happened.

A smart computerized medicinal substance storage room or rooms of the factories, farms, processing facilities, storage facilities or laboratories where controlled or regulated substances such as cannabis or opiates are kept/stored may also be linked to and monitored by the smart container centralized computer network 466 and data cloud system 512. The smart computerized medicinal substance storage room and configured with the smart container centralized computer network 466 and data cloud system 512. The smart container centralized computer network 466 and data cloud system 512 may include A.I./Artificial Intelligence Software that may be designed to recognize the smart medicinal substance storage room as a “fixed” or “non-portable” child resistant medicinal substance container. The smart computerized medicinal substance storage room “Fixed Smart Container” may also be configured with the same elements that make up the medicinal substance monitoring system 499. These elements may include but are not limited to magnetic locking system 462, High-Definition camera 461, light sensor 476, motion/vibration sensor 477, sound sensor, touchscreen user interface 457 7 458, memory 467, sliding cartridge 320 321, programmable LED lights 460 with colors that have assigned meanings (the assigned meanings relevant to identifying or classifying cannabis or other regulated substances “VIP Color”). In other words, the smart computerized medicinal substance storage room paired and on the smart container centralized computer network 466 and data cloud system 512 is recognized and controlled as a large “fixed” container. (NOTE: The smart containers may be smart maritime cargo international freight shipping containers, smart commercial portable storage pod containers, Smart Eighteen-Wheeler Truck Trailer Containers, or Smart Locomotive Containers/Train Car. Lithium Ion Battery Powered Autonomous Self Driving Eighteen Wheelers or Semi-Trucks like those manufactured by Tesla Motors may haul and transport the smart electronic truck trailer/container. The Smart Eighteen-wheeler or other DOT Department of Transportation) regulated vehicles may also be connected to and monitored by the smart container centralized computer network 466 and data cloud system 512). By configuring the child resistant smart containers in all their different styles, variations, and applications to lock and unlock by processing data received and sent by the sensors 476, 477, and 478. The software application 471, when downloaded onto the portable smart computing device 471, allows the user enter input touchscreen 464 and the computing device analog buttons to control the programmable LED lights 462. (Note: Usually the computing device analog buttons are located on the sides of the portable smart computing device and may function as the volume controls and or On and Off buttons of the portable smart computing device 462). For example, a user may press a volume control button on its upper side to brighten the programmable LED lights 462 and press the lower side of the volume button to dim the programmable LED lights 462. The Software application 471 provides a Light Color menu page. The Light color Menu page features a selection of Colors that Programmable LED Light 462 may turn. A user may be able to input a command by way of tapping an icon displayed on the touchscreen 464 colored a color and displayed on the Light color menu page, and send a signal to the Processor 468 to read and execute a command to change the programmable LED lights 462 to the Color. The software application 471 may provide a light pattern control feature.

The child resistant medicinal substance container 300 can save battery power. The child resistant medicinal substance container 300 can be programmed to lock the container lid 302 when an unauthorized individual is in the vicinity of The child resistant medicinal substance container 300 and triggers any of the sensors 476, 477, 478. The child resistant medicinal substance container 300 may be program to be in sleep mode/Standby mode and stay in sleep mode until an unauthorized individual is in the vicinity of the container, interacts with the container or when the sensors 476, 477, 478 are triggered and send a signal to the processor 468 and the processor 502 which send signal to the electromagnetic locking system to draw current or voltage from the battery 481 to the electromagnetic lock and armature plate to lock the container 300. A user or medical professional can monitor the location of, or even lock or unlock the child resistant medicinal substance container 300 remotely because of the micro-GPS module 463, and the Wi-Fi module 480 which are connected to the smart container centralized computer network 466. The medicinal substance monitoring system 499 may have an alarm application software integrated with a clock and calendar app to remind a patient or their caretakers of certain events. These reminders or alerts may be sent via an audible method using the small micro speaker 472. These reminders or alerts may be a SMS, a text message, a email or any other known method of communication known in the art to be used by the peripheral smart mobile computing device 462 The calendar app of the medicinal substance container may be compatible and sync with a popular calendar application software systems used or created by Amazon, Google, Samsung, Apple, or Microsoft systems or their devices. For example, The Artificial Intelligence Software A.I. voice assistants of the companies named above like Alexa, Amazon's smart home system assistant may be configured to sync send and receive data to and from the centralized smart container computer network system 466 and the computer systems 499 of the paired smart medicinal substance container 300 and alert the patient about an event through Alexa's speaker systems also. Other AI Voice systems like Apple's Siri, Samsung's Bixby, Microsoft's Cortana, or Googles Assistant may receive and convey information sent by the centralized smart container computer network system 466 audibly through their respective speaker systems. These voice assistants may be licensed from their respective owners to use their recognized voices as the voice of the smart lockable handheld medicinal substance container 300 that is paired the centralized smart container computer network system 466. By designing the smart medicinal substance containers software 471 to use or sync with the arts leading calendar apps and voice assistants, the chance of a patient or user receiving the alerts increases because, a patient can sync the smart medicinal substance container's software 471 with the calendars they are already using daily on their personal and or cooperate supplied smart mobile devices and applications. These alerts may be used also to remind a patient that it is time to consume their medication. They may also notify the patient, or any party connected to the security network that the container is locked or unlocked, may be regarding smart the container's location, may be regarding the change of the weight or volume of the substance in a smart container or a cartridge that is inserted in the smart container, may notify a party connected to the network that container will be locked or unlocked at a scheduled time. (Note: The audible voice may be programed to be the voice of a celebrity or other voice.)

1. A multi-chambered medicinal substance container with a container body, a container lid, and a medicinal substance cartridge assembly having a first medicinal substance cartridge, and a second medicinal substance cartridge comprising,
the container body,
the container lid,
the medicinal substance cartridge assembly including the first medicinal substance cartridge and the second medicinal substance cartridge, the medicinal substance cartridge assembly designed so that the second medicinal substance cartridge is slidably inserted into the first medicinal substance cartridge, and the whole medicinal substance cartridge assembly can be slidably inserted into the container body,
wherein the container lid is secured to the container body via a child resistant feature.

2. A multi-chambered medicinal substance container as recited in claim 1 wherein,
the container body is elongated and cylindrically shaped.

3. A multi-chambered medicinal substance container as recited in claim 1 wherein,
the medicinal substance cartridge assembly is elongated.

4. A multi-chambered medicinal substance container as recited in claim 3 wherein,
the first medicinal substance cartridge is designed in a cylindrical shape.

5. A multi-chambered medicinal substance container as recited in claim 1 wherein,
the multi-chambered medicinal substance container is designed to retain a cannabis product derived from a cannabis plant.

6. A multi-chambered medicinal substance container as recited in claim 2 wherein,
the multi-chambered medicinal substance container is designed to retain a cannabis product in the first and second medicinal substance cartridges.

7. A multi-chambered medicinal substance container as recited in claim 3 wherein,
first medicinal substance cartridge is designed to retain cannabis flower, and second medicinal substance cartridge is elongated and designed with at least designed to retain at least one pre-rolled smokable article.

8. A multi-chambered medicinal substance container as recited in claim 4
wherein, the pre-rolled smokable article is a pre-rolled cannabis cigarette.

9. A multi-chambered medicinal substance container as recited in claim 5 wherein,
the second medicinal cartridge is designed with a medicinal substance tube configured to retain the at least one pre-rolled cannabis cigarette.

10. A multi-chambered medicinal substance container as recited in claim 6
wherein, the second medicinal substance cartridge has a cartridge head.

11. A multi-chambered medicinal substance container as recited in claim 7 wherein,
the second medicinal substance cartridge head provides at least one cartridge head hole configured to be an opening to the medicinal substance tube when the medicinal substance tube is secured to the cartridge head.

12. A multi-chambered medicinal substance container as recited in claim 8 wherein,
the second medicinal substance cartridge head provides a head port designed to receive the at least one medicinal substance tube in a secured way.

13. A multi-chambered medicinal substance container as recited in claim 9 wherein,
the head port is threaded on the inside and is designed to receive the medicinal substance tube by way of the medicinal substance tube being screwed into the port via threads on the outside wall of the upper end of the medicinal substance tube.
